(19) United States
(12) Patent Application Publication
(10) Pub. No.: US 2026/0051366 A1
(43) Pub. Date: Feb. 19, 2026

(54) METHODS AND SYSTEMS FOR IDENTIFYING GENE REGULATORY ELEMENTS AND ALTERING GENE REGULATION AND EXPRESSION

(71) Applicant: The Trustees of Columbia University in the City of New York, New York, NY (US)

(72) Inventors: Xi Fu, New York, NY (US); Raul Rabadan, New York, NY (US)

(21) Appl. No.: 19/307,934

(22) Filed: Aug. 22, 2025

Related U.S. Application Data
(63) Continuation of application No. PCT/US2024/017064, filed on Feb. 23, 2024.
(60) Provisional application No. 63/486,855, filed on Feb. 24, 2023.
(60) Provisional application No. 63/541,164, filed on Sep. 28, 2023.

(51) Int. Cl. G16B 25/10; G16B 30/00; G16B 40/20; G16B 40/30

(52) U.S. Cl. CPC G16B 25/10; G16B 30/00; G16B 40/20; G16B 40/30

(57) ABSTRACT

The present disclosure provides methods and systems for identifying transcriptional regulatory modules (e.g., in non-coding portions of the genome), predicting gene regulation and expression, e.g., effects of non-coding mutations or chromosome rearrangements on the regulation and expression of the target genes, and designing and using modified regulatory sequences.

### CROSS-REFERENCE TO RELATED APPLICATIONS

This application is a continuation of International Application No. PCT/US2024/017064, filed Feb. 23, 2024, which claims the benefit of U.S. Provisional Application Nos. 63/486,855, filed Feb. 24, 2023, and 63/541,164, filed Sep. 28, 2023, the contents of which are herein incorporated by reference in their entirety.

### STATEMENT REGARDING FEDERALLY SPONSORED RESEARCH OR DEVELOPMENT

This invention was made with government support under CA253126, awarded by the National Institutes of Health. The government has certain rights in the invention.

### FIELD

The present invention relates to methods and systems for identifying transcriptional regulatory modules (e.g., DNA sequences in non-coding portions of the genome), predicting gene regulation and expression, e.g., effects of non-coding mutations or chromosome rearrangements on the regulation and expression of the target genes, and designing and using modified regulatory sequences.

### SEQUENCE LISTING STATEMENT

The content of the electronic sequence listing titled COLUM-41568.601.xml (Size: 2,860 bytes; and Date of Creation: Feb. 21, 2024) is herein incorporated by reference in its entirety.

### BACKGROUND

Transcriptional regulation is influenced by a complex network of signaling molecules, transcription factors, epigenomic marks and chromatin availability, and DNA sequences near and far from the target gene each of which playing a role on the resulting level of target gene expression. Understanding how transcriptional regulatory networks influence gene expression and being able to predict gene expression from aspects of those networks is essential to elucidate disease and other biological mechanisms, predict non-coding mutation and chromatin translocation effects, and eventually automate the diagnosis and treatment of human diseases and conditions, rationally modify gene expression, and rationally design synthetic biological systems.

### SUMMARY

In some embodiments, provided herein are methods for identifying genetic regulatory modules. In some embodiments, the methods comprise at least one or all of: obtaining sequence features of genomic regions and expression data for one or more target genes from a biological sample or database; determining interaction between sequence features; generating a score representing the effect on expression of a target gene for the sequence features or combinations thereof; and identifying regulatory modules based on the score. In some embodiments, the methods further comprise assigning a regulatory module as a transcriptional enhancer or transcriptional repressor based on the score.

In some embodiments, the sequence features comprise chromatin accessibility data, transcription factor binding motif data, nucleotide sequences, or a combination thereof. In some embodiments, the chromatin accessibility data comprises nucleotide level accessibility data. In some embodiments, the chromatin accessibility data comprises Assay for Transposase-Accessible Chromatin (ATAC) data.

In some embodiments, the expression data comprises bulk RNA-seq data, single cell RNA-seq data (scRNA-seq) or single nucleus RNA-seq data (snRNA-seq).

In some embodiments, wherein the sequence features and/or expression data are derived from a single cell. In some embodiments, the sequence features and/or expression data are derived from a single cell type. In some embodiments, the sequence features and/or expression data features are derived from bulk cell data.

In some embodiments, the genomic regions comprise regions within 20 kilobases of the transcription start site of a target gene. In some embodiments, the genomic regions comprise regions greater than 1 Megabase from the transcription start site of a target gene. In some embodiments, the genomic regions are within 2 Megabases of the transcription start site of a target gene.

In some embodiments, determining the interaction between sequence features comprises using a deep learning model. In some embodiments, the deep learning model comprises a transformer model.

In some embodiments, the methods further comprise generating a score representing the effect on target gene expression for one or more identified regulatory modules with a machine learning model. In some embodiments, the machine learning model comprises a language model.

In some embodiments, the methods further comprising generating a score for one or more modified regulatory modules, wherein the modified regulatory modules comprise a modification to one or more features of the identified regulatory module. In some embodiments, the modification comprises one or more nucleic acid substitutions, deletions, and/or insertions in a sequence of a regulatory module.

Also provided herein are methods for predicting target gene regulation and expression. In some embodiments, the methods comprise identifying regulatory modules for a target gene; and generating a score representing the effect on target gene expression for one or more identified regulatory modules with a machine learning model. In some embodiments, the machine learning model comprises a language model.

In some embodiments, the methods further comprising generating a score for one or more modified regulatory modules, wherein the modified regulatory modules comprise a modification to one or more features of the identified regulatory module. In some embodiments, the modification comprises one or more nucleic acid substitutions, deletions, and/or insertions in a sequence of a regulatory module.

In some embodiments, identifying regulatory modules for a target gene uses the methods disclosed herein.

In some embodiments, training for the machine learning model is selected from the group consisting of unsupervised learning, self-supervised, semi-supervised learning, transfer learning and combinations thereof. In some embodiments, training comprises at least one or all of: obtaining sequence features of genomic regions and expression data for a plurality of target training genes from a single cell or cell line; determining interaction between sequence features; and identifying putative regulatory modules based on the effect on expression for the sequence features or combinations thereof.

In some embodiments, the sequence features comprise chromatin accessibility data, transcription factor binding motif data, nucleotide sequences, or a combination thereof. In some embodiments, the chromatin accessibility data comprises nucleotide level accessibility data. In some embodiments, the chromatin accessibility data comprises Assay for Transposase-Accessible Chromatin (ATAC) data.

In some embodiments, the expression data comprises single cell RNA-seq data (scRNA-seq) or single nucleus RNA-seq data (snRNA-seq).

In some embodiments, the genomic regions comprise regions within 20 kilobases of the transcription start site of a target gene. In some embodiments, the genomic regions within 2 Megabases of the transcription start site of a target gene.

In some embodiments, determining interaction between sequence features comprises using a deep learning model. In some embodiments, the deep learning model comprises a transformer model.

In some embodiments, the target gene is from the same or different cell or cell-type as the single cell or cell line used in the training.

In some embodiments, the methods further comprise designing, and optionally conducting, one or more gene editing experiments to generate a modified regulatory module. In some embodiments, the gene editing experiments comprise methods based on CRISPR-Cas, transcription activator-like effector nucleases (TALENs), zinc-finger nucleases (ZFNs), and homing endonucleases or meganucleases.

Further provided are non-transitory computer-readable media storing instructions, that when executed by one or more processors performs operations to carry out the steps of the disclosed methods and systems comprising one or more processors and the computer readable media.

Other aspects and embodiments of the disclosure will be apparent in light of the following detailed description and related figures.

### BRIEF DESCRIPTION OF THE FIGURES

FIG. 1 is a schematic of an exemplary experimental pipeline for the methods disclosed herein. Multi-omic single-cell data (e.g., RNA-seq and ATAC-seq data) from human cells is collected from public website or acquired through experiments and used to train a machine learning model that can accurately predict gene expression in that cell type from chromatin accessibility data. The model is used toward the inference of interactions between regulatory elements (RE), transcription factors (TF), and target genes. The identified transcriptional regulatory modules provide a framework to predict the effects of non-coding mutations or chromosome rearrangements on the regulation and expression of the target genes.

FIG. 2 is an example of in silico genome editing using the methods disclosed herein. The transcriptional impact of two TERT promoter mutations (C228T and C250T) is predicted using the methods herein. For C228T, the method identified that the mutation replaced a SMAD binding site with a ETS1 binding site. While SMAD is both known and predicted to be a repressor of TERT gene, and ETS1 is both known and predicted to be an activator of TERT. Thus, an increased expression of TERT caused by the two promoter mutations comparing other mutations in the loci was predicted.

FIGS. 3A-3B are comparisons of methods of the present invention (GRAD) to other models of identifying experimentally validated regulatory elements in fetal erythroblast cell line, overall (FIG. 3A), with regions far (>20 kbp) from the transcription start site (TSS) (FIG. 3B), and with regions close (<20 kbp) from TSS (FIG. 3C) for BCL11 Transcription Factor A (BCL11A), MYB proto-oncogene, transcription factor (MYB), KLF transcription factor 1 (KLF1), and hemoglobin subunit beta (HBB).

FIGS. 4A-4C show the use of the methods for identifying the regulatory elements, upstream regulator, or regulatory nucleotides for BCL11A gene. FIG. 4A shows the location of BCL11A on chromosome 2 and the regions identified for transcriptional effects using erythroblast Assay for Transposase-Accessible Chromatin (ATAC) data, fetal primary cell data, and HUDEP, publicly available fetal erythroblast cell line, ATAC data, public fetal erythroblast cell line data. The effect on expression is the prediction or Gradient (GRAD) score and HUDEP Locus Score is the regulatory score defined by base editing screening. FIG. 4B is a heat map of the regulatory effect of various motifs. Column ID 414 is the BCL11A erythroid enhancer. Column ID 450 is the BCL11A promoter. The disclosed methods identified the important motif (GATA-Ebox/SNAI2) for the BCL11A erythroid enhancer. FIG. 4C shows an in-silico base-editing experiment performed using the disclosed methods to predict mutation effects on BCL11A expression more accurately, as compared to Enformer, DeepSEA, GERP, CADD, and random baselines. FIG. 4D shows an example of in-silico CRISPR to model transcriptional impact of recurrent chromatin rearrangement of MGMT gene in glioblastoma.

FIGS. 5A-5D are an overview of the curated training dataset. FIG. 5A is a schematic of single-cell ATAC-seq atlas defining a unified accessible region pool. Different cell types contain a different sampling from the pool. FIG. 5B is a schematic of organ-level train-test split and how samples are defined as a consecutive set of accessible regions in a specific cell type. FIG. 5C is a schematic of motif and sequence representation of regions and how they are combined across multiple regions in one training sample. Sequences shown in right panel are SEQ ID NOs: 1 (left-hand) and 2 (right-hand). FIG. 5D is a correlation map across 12 different cell types, as labeled.

FIG. 6A is a schematic of GET by unsupervised transfer learning for out-of-distribution gene expression prediction. In the unsupervised stage, randomly masked regions embeddings are taken as input and multiple region-wise attention layers are applied to recover the masked regions by learning the general cis- and trans-interactions between regulatory elements and transcription factors. With the pre-trained weights and a task head, GET is transferred to the finetune dataset for gene expression prediction. Sequences shown in top right panel are SEQ ID NOs: 1 (left) and 2 (right). The architecture of an exemplary model of GET is shown in FIG. 6B. Enformer architecture is shown alongside for comparison.

FIG. 7 is visualization results of Transcription Start Sites scatter plots for Enformer, GET (w/o stage 1), GET (w/o stage 2), and GET. GET with both stages achieves the highest Pearson R score between predictions and targets.

FIG. 8 is quantitative results of out-of-distribution gene expression prediction on 12 leave-one-out cells. For comprehensive evaluation, the averaged score was reported using 3 different random seeds.

FIG. 9 is attention maps of transcription regulatory interactions for gene expression prediction. Top Row: unsupervised attention weights in stage 1; Bottom Row: transferred attention weights in stage 2. The locations of TSSs are marked with red rectangles.

FIGS. 10A-10E show the GET model's universal applicability and exceptional accuracy as a foundational transcription model. FIG. 10A shows that GET derives transcriptional regulatory syntax (pretrain) from chromatin accessibility data across hundreds of cell types, furnishing reliable predictions (finetune) of gene expression in both seen and unseen cell types. The model's broad applicability and comprehensibility allow for zero-shot prediction of lentiMPRA measurements, extensive identification of cell-type-specific regulatory elements and upstream transcription factors (transcription factors), universal embedding of regulatory information, and facilitating causal understanding of transcription factor-transcription factor interactions. FIG. 10B is an illustration of a training scheme of GET. The input of GET is a peak (accessible region) by transcription factor (motif) matrix derived from human single cell (sc) ATAC-seq atlas, summarizing regulatory sequence information across a genomic locus of more than 2 Mbps. Through self-supervised random mask-prediction pretrain of the input data across more than 200 cell types, GET learns transcriptional regulatory syntax (p). Finetuned on paired single cell ATAC-seq/RNA-seq data, GET learns to transform the regulatory syntax to gene expression even in leave-out cell types.(f.p). FIG. 10C is benchmark GET prediction performance on unseen cell types (Fetal astrocyte). Each point is a gene. Color represents normalized chromatin accessibility in TSS. Gene activity is a score widely used in modern scATAC-seq analysis pipeline. Top correlated cell type is the training cell type whose observed gene expression has the largest correlation with Fetal astrocyte, in this case Fetal inhibitory neuron. Mean cell type is the mean observed gene expression across training cell types. Dash line represents linear fits. FIG. 10D is an exemplary visualization of observed expression (top, log 10TPM), GET prediction (mid, log 10TPM) and chromatin accessibility (bottom, log 10CPM) of the BCL11A locus in Fetal erythroblast. Positive (negative) values represent expression on positive (negative) strand on hg38. FIG. 10E is a graph of GET trained on fetal cell types generalize to adult cell types without retraining, outperforming most correlated cell type baseline. X axis showing R2 score between GET prediction in adult cell types and observed expression in most similar fetal cell types. Y axis showing R² score between GET prediction and observed expression in the adult cell type.

FIGS. 11A-11E show transfer learning adapts GET to new platforms and measurements. FIG. 11A is a schematic illustration of transferring GET to lymph node 10× multiome dataset. FIG. 11B shows finetuned GET accurately predicts expression in training and leave-out evaluating cell types. FIG. 11C is a schematic workflow of lentiMPRA experiments and in silico lentiMPRA using GET model finetuned on K562 multiome data. FIG. 11D is readout distribution of lentiMPRA (log 2RNA/DNA) and GET prediction (mean expression across genomic insertions) for different types of elements. FIG. 11E shows benchmark GET lentiMPRA prediction against Enformer on random subset of elements.

FIGS. 12A-12G show that the GET model identifies cell-type-specific regulator and cis-regulatory elements. FIG. 12A is a schematic of a case study for identifying cis-regulatory elements (CRE) and regulators controlling a phenotype, fetal hemoglobin (HbF) level. Four genome-wide association loci (BCL11A, MYB, NFIX, HBG2) have been subjected to genome editing in a previous study, providing the labels for GET benchmark. Region/motif contribution score for each gene can be computed using GET model. FIG. 12B shows that GET identifies GATA motif in erythroid-specific enhancer that upregulates BCL11A, an HbF repressor. Top: motif contribution score for BCL11A expression in the erythroid-specific enhancer. Mid: gRNA enrichment score (HbFBase). Higher score means enrichment in high HbF cells, which implies these edits disturb a cis-regulatory element or regulator binding site that can upregulate BCL11A. Bottom: single cell ATAC-seq signal and peak from Fetal erythroblast. FIG. 12C is a genome track of inferred CREs for BCL11A, MYB, NFIX and HBG2. From top to bottom: HbFBase: the gRNA enrichment score from base-editing experiments. GET: GET-infered region importance score (Methods). Enformer: Enformer-infered region importance score. ABC: Activity-by-contact prediction collected from the original base-editing study. ATAC/HUDEP-2: Chromatin accessibility track of HUDEP-2, the erythroblast cell line used in base-editing study. ATAC/Fetal Erythroblast: Chromatin accessibility track of Fetal erythroblast, used in the training of GET. HiChIP/HUDEP-2: H3K27ac HiChIP track of HUDEP-2 cell line. FIG. 12D is benchmark results of GET against existing methods and baselines at two HbFBase cutoffs. Left shows results for all enhancer-promoter pairs. Right shows results for only distal enhancer-promoter pairs with distance larger than 100 kbp. AUPR: area under precision and recall curve. FIG. 12E is the predicted top 3 three regulators (motifs) for BCL11A, NFIX and HBG2. Similar sequence patterns are highlighted with color shades. FIG. 12F shows GATA downstream targets inferred by GET (top 10% motif score) showing functional enrichment in Hemopoiesis. Scatterplot shows predicted gene expression (X-axis) and GATA-motif score (Y-axis) for GATA-targeted genes with predicted expression larger than one. All transcription factors among these genes are labeled in the plot, where those involved in Hemopoiesis are highlighted in red color. FIG. 12G shows the correlation between motif contribution (y-axis) in ‘Fetal Erythroblast 1’ and the predicted target gene expression change (x-axis) between ‘Fetal Erythroblast 1’ and Fetal HSC. Four motifs relevant to erythroid differentiation are shown.

FIGS. 13A-13H show GET captures regulatory information across cell types and informs casual transcription factor-transcription factor interaction. FIG. 13A is a schematic of the workflow to collect and visualize cross-cell-type regulatory embedding. FIG. 13B shows that the cross-cell-type regulatory embedding reveals cell-type specificity in transcription regulation. Subsampled embedding from Fetal astrocyte (blue) and two Fetal erythroblast (yellow and brown) cell types are visualized with UMAP. FIG. 13C is Louvain clustering of subsampled embedding in FIG. 13B. Note that cluster 2 is the astrocyte specific cluster. FIG. 13D is gene ontology enrichment of genes in cluster 2. Showing astrocyte-relevant terms and astrocyte marker genes e.g., NFIA, GLI3. FIG. 13E is GET motif contribution Z-scores (Methods, red means higher score comparing to other clusters) for each clusters. Cluster 2 has elevated NFI/1 and NFI/2 motifs, which correspond to the NFI family transcription factors. FIG. 13F shows that causal discovery using the GET motif contribution matrix identifies transcription factor-transcription factor interaction. Physical interactions from STRING databases are used as a benchmark to calculate the concordance. FIG. 13G is an example causal neighbor graph showing interactions (edges) between motifs (nodes). Edge weights means interaction effect size. Edge directions marks casual direction. Blue and red edge color marks negative or positive effect size. Node color marks community detected on the full causal graph. In-community edges are marked by reduced saturation. FIG. 13H is a benchmark of the concordance of inferred transcription factor-transcription factor interaction using different methods with physical interactions from the STRING database. X axis marks different cutoffs of retained interaction in percentile of 79,242 total possible interactions. Y axis marks the ratio of selected interactions that is also marked as interacted in STRING. Green line marks the random selection background. Orange line marks the result of selection using motif-motif contribution score correlation. Blue line marks the causal discovery result. Shaded area marks standard error across 5 bootstraps. The star marks the result from a recent mass-spectrometry-based transcription factor-transcription factor interaction atlas.

FIGS. 14A-14K show structural properties of inferred transcription factor-transcription factor interactions through GET causal discovery. FIG. 14A is catalogs of transcription factor-transcription factor interactions. Direct interaction includes homodimer, intra-family heterodimer or inter-family heterodimer. Cofactor-mediated interaction may include both cooperative and competitive binding. FIG. 14B shows a pLDDT plot for TFAP2A and ZFX, showing correspondence between high pLDDT regions and known protein domains (red rectangles). FIG. 14C is a predicted monomer structure of ZFX, showing DNA binding domain (DBD, grey) and intrinsically disordered region (IDR, red). FIG. 14D is a predicted structure of TFAP2A structured domains and ZFX IDR. Red and blue color marks negative and positive electrostatic surfaces. FIG. 14E is a molecular dynamics simulation of TFAP2A-ZFX IDR (red) or ZFX IDR monomer (purple). Collapsed structure in ZFX IDR monomer is highlighted in rectangle. FIGS. 14F-14G show the correlation between pLDDT and residue RMSD across the simulation trajectory of ZFX IDR in the complex structure. Visualized in scatter plot and line plot across the protein sequence. Yellow and blue shades in the line plot highlight beta sheets or alpha helices.

FIG. 14H shows a pLDDT plot for EP300, highlighting TAZ1 and TAZ2 transcription factor interacting domain. Region of interest (red) and domain (green) marks annotated regions on UNIPROT. Low pLDDT regions are highlighted in gray shades. FIGS. 14I-14K predict structural interaction between SNAI1 N-terminal and EP300 TAZ2 domain (FIG. 14I), SNAI1 N-terminal and EP300 TAZ1 domain (FIG. 14J), and RELA C-terminal and EP300 TAZ1 domain (FIG. 14K). Red and blue color marks negative and positive electrostatic surfaces.

FIGS. 15A-15F show that GET identifies a cell type specific transcription factor-transcription factor interaction affected by a cancer-associated germline variant. FIG. 15A is a pLDDT plot for PAX5. Showing three mutational hotspots: V26G, P80R, G183S/V/A and two frameshift hotspots. Region annotations from UNIPROT are shown in the figure as ‘region of interest.’ FIG. 15B shows B-cell specific motif interactions of PAX/2. PAX5 is the highest expressed transcription factor with PAX/2 motif. RORA is the highest expressed transcription factor with the NR/3 motif. Color scheme follows FIG. 13G. FIG. 15C shows a predicted multimer structure of PAX5 IDR and RORA NR domain. Showing contacts around G183 and R225. FIG. 15D is a Venn diagram of identified PAX/2 and NR/3 specific and common regulatory targets using GET gene-by-motif importance matrix. FIG. 15E is enrichment analysis using B-cell associated gene sets in Shah et al. (Nature Genet 24, 1226-1231 (2013); top) and biological process gene ontology gene sets (bottom). Results for the PAX/2-NR/3 common genes are shown in this figure. Results for PAX/2 or NR/3 specific genes are shown in FIG. 24. FIG. 15F is Spearman correlation between PAX5 (PAX/2), RARA (NR/3) and the average expression of PAX/2-specific (blue), NR/3 specific (orange) and common (light brown) target genes in B-ALL patients without PAX5 somatic coding mutations.

FIG. 16 is the architecture of GET in comparison with Enformer

FIG. 17A shows pairwise correlation between human astrocytes in different culturing conditions. FIG. 17B shows log fold change prediction performance of GET between unseen (Astrocytes) and seen (other) cell types.

FIG. 18A shows prediction performance of GET transferred to predict ATAC counts in open chromatin of K562, showing scatter plot of prediction vs observed and standard deviation of prediction vs mean prediction for the MPRA sequence elements. FIG. 18B shows benchmarking standard deviation of MPRA zeroshot prediction across random insertions. FIG. 18C shows detailed ablation of expression and ATAC component of GET prediction.

FIG. 19A is cis-regulatory region prediction of MYB and HBG2 loci. FIG. 19B is per-gene AUPR benchmark of cis-regulatory region prediction.

FIG. 20 shows the correlation between mean transcription factor expression in a motif cluster versus the mean expression of their target genes predicted by GET across fetal cell types.

FIG. 21A is the correlation between motif clusters using showing potential coregulation. FIG. 21B is the top correlated motif pairs have significantly larger functional similarity. X axis iscosine similarity computed on term (motif clusters) frequency-inverse document (Gene Ontology biological process) frequency (transcription factor-IDF) matrix. FIG. 21C is a casual motif-motif interaction network inferred from all cell types. Edges with absolute effect size smaller than 0.1 and isolated nodes are removed for visualization purposes. Node colors represent communities. Edge weights represent causal effect sizes. Edge colors represent positive (orange) or negative (blue) effects.

FIGS. 22A-22C show the overall network statistics of causal motif-motif network. FIG. 22A is the out-degree distribution across cell-type-specific causal networks at two different absolute edge weight cutoffs. Error bar shows standard deviation across cell types. FIG. 22B is the out-degree distribution of With-ATAC GET model trained using fetal data. FIG. 22C is the betweenness centrality and absolute effect on accessibility or ZFX across cell-type-specific causal networks at edge cutoff 0.01.

FIG. 23A is prediction performance of ‘intra-family binder’ using different multimer structure prediction confidence scores. Left: ROC curve with x-axis showing false positive rate and y-axis showing true positive rate. Right: PR curve with x-axis showing recall and y-axis showing precision. mean_plddt: average predicted Local Distance Difference Test (pLDDT) score across all residues. pae: predicted Aligned Error across all inter-chain interactions. pdockq: predicted DockQ metric using interface pLDDT. pdockq_pae: multiplication of pDockQ and pAE. FIG. 23B is a comparison of Alphafold2 predicted TFAP2A dimer structure (pink) with crystal structure of TFAP2A (yellow)-DNA (green) complex. FIG. 23C shows the change of number of hydrogen bonds in TFAP2A-ZFX IDR complex or ZFX IDR monomer across simulation trajectory. FIG. 23D shows anti-correlation between pLDDT and average RMSD across trajectory along TFAP2A or ZFX chain. FIG. 23E is a predicted multimer structure of EGR1 IDR-ZFX IDR. FIG. 23F is a predicted multimer structure of ZEB1 C-terminal and ESR1 NR domain.

FIGS. 24A-24B show gene enrichment analysis of NR/3-specific (orange) and PAX/2-specific (blue) target genes using published B cell related genesets (FIG. 24A) and gene ontology biological processes (FIG. 24B). FIG. 24C is motif visualization and comparison. From top to bottom: LHX6 (a neuronal lineage TF), PAX5 motif in PAX/1 motif cluster, PAX5 core motif and reverse complement, PAX5 motif in PAX/2 motif cluster, RORA and RARA motifs in NR/3 motif cluster. FIG. 24D is Spearman correlation between two NR/3 transcription factors (NR4A1, RORA) and the average expression of PAX/2-specific (blue), NR/3 specific (orange) and common (light brown) target genes in B-ALL patients without PAX5 somatic coding mutations.

FIG. 25 is zero-shot application of astrocyte-leave-out Fetal-data-trained model (nucleotide-level input version) to an independently processed Glioblastoma dataset, demonstrating universal adaptability across datasets.

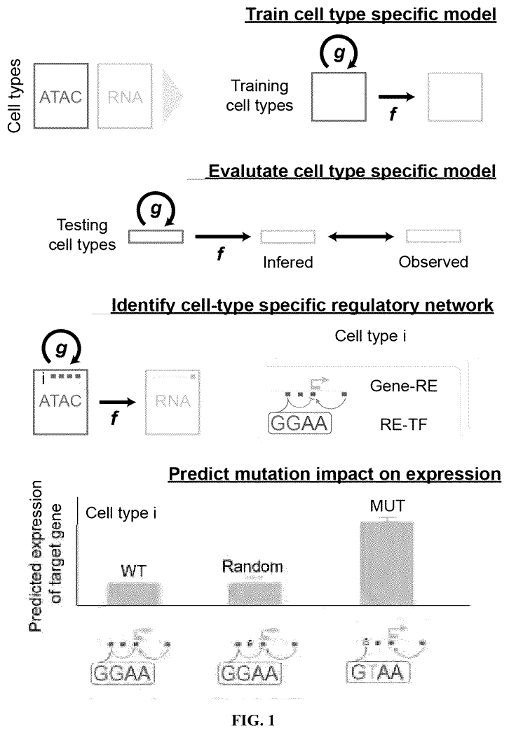

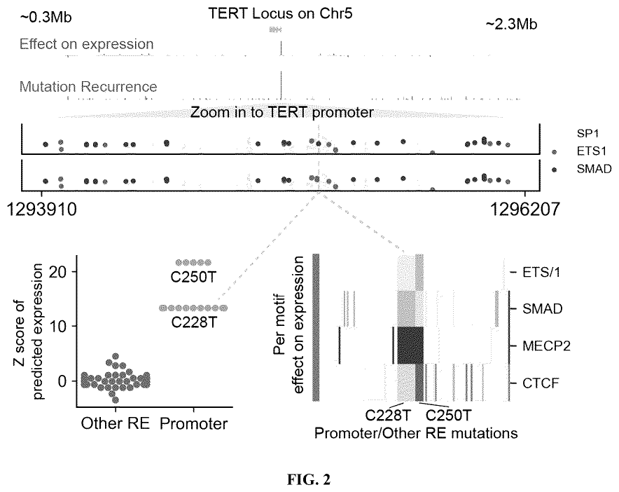

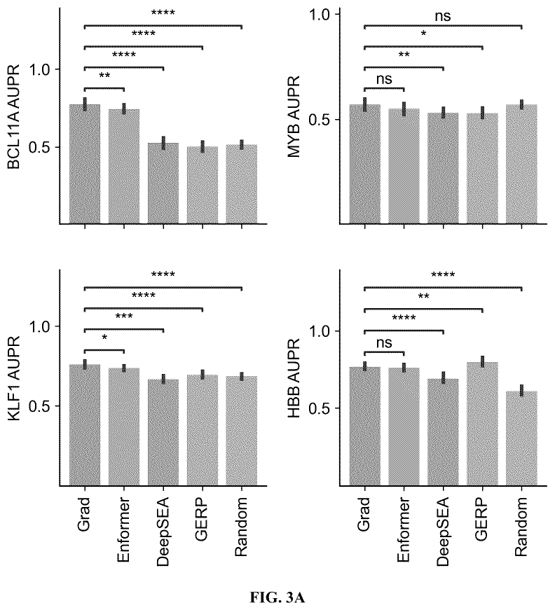

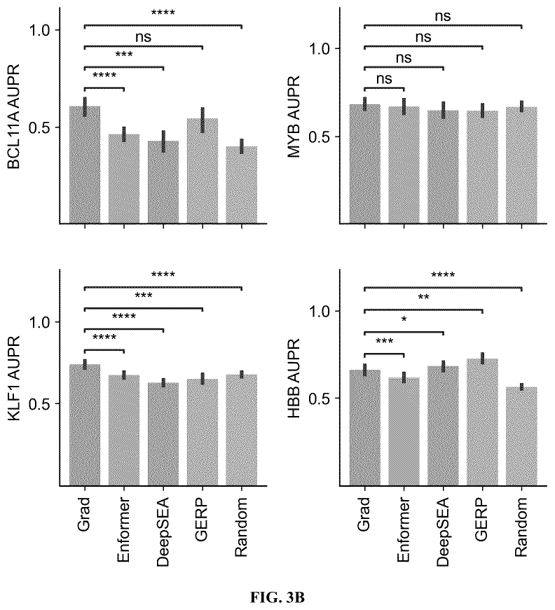

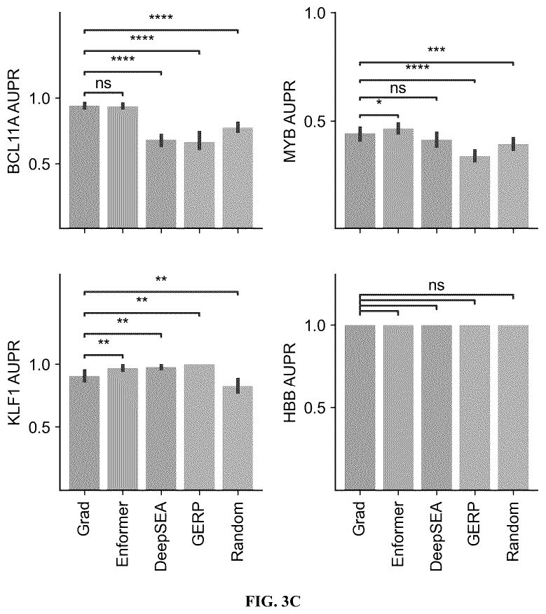

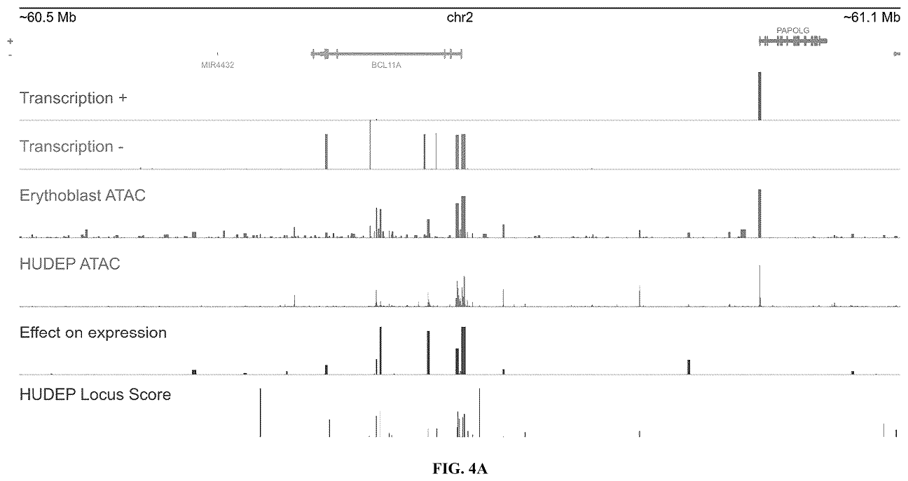

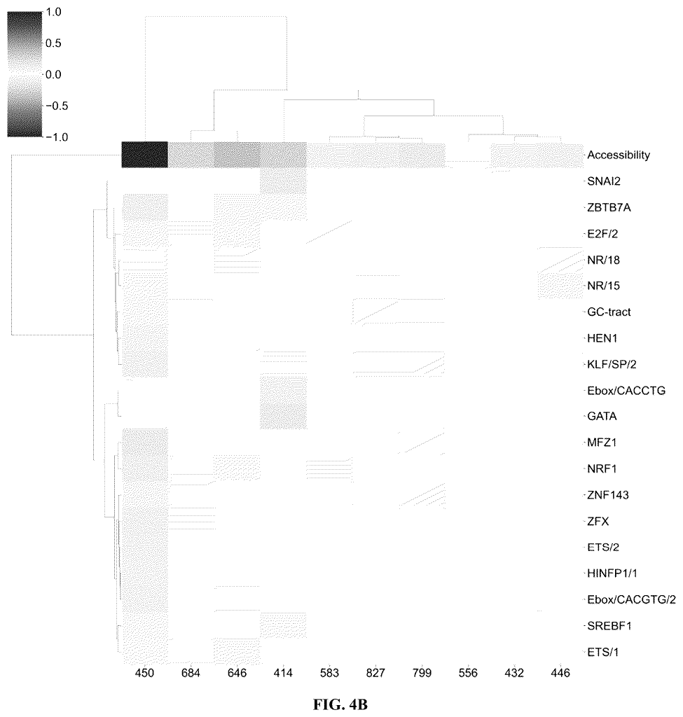

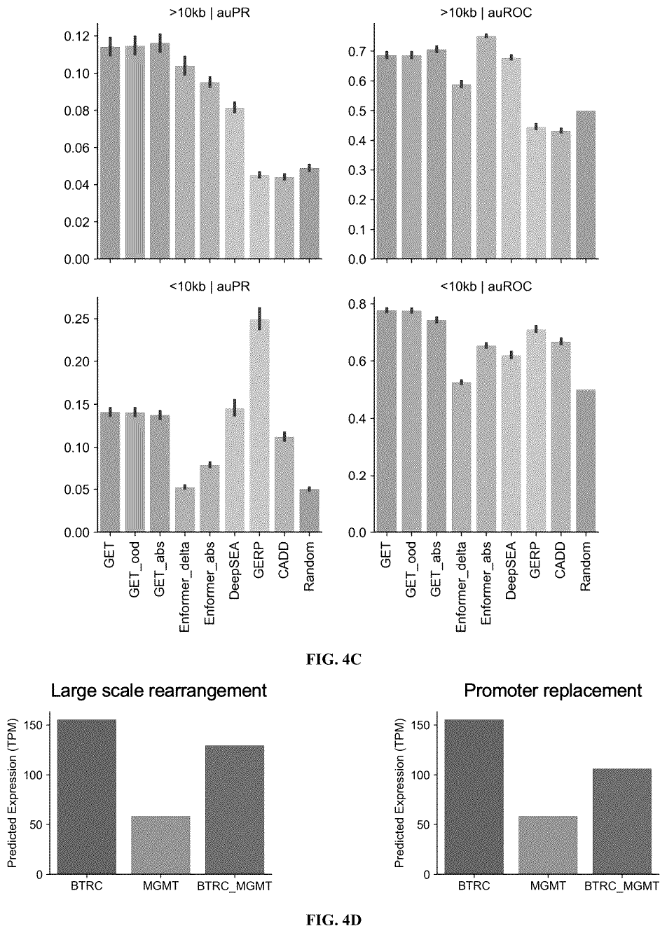

### DETAILED DESCRIPTION

The task of gene expression prediction based on epigenomic marks like histone modification or chromatin accessibility has been explored repeatedly over the past decades. Most previous models predict the expression value of one or multiple genes using sequence features and/or epigenomic signals in the same cell types. A large number of conventional methods, including Basenji2 and Expecto, have relied on convolutional neural networks to learn patterns of expressed genes. The input sequence length has been increased over the course of several years with the increased capacity of the model to associate a gene with a distal regulatory element but is still limited for many long-range interactions that happen in the cell. Also, many models (e.g., Expecto) perform the cell-type specific expression prediction by training a separate model for each cell type, leading to difficulty in learning a generalizable representation of the regulatory mechanism. Enformer, a more recent model, combines dilated CNN and transformer architecture as well as multi-head output to predict gene expression and epigenomic marks, and gain some performance improvements against conventional methods. Overall, the present methods do not allow cross cell-type prediction, are limited in cross-organism predictions, use bulk cell data which does not capture cell-type or timepoint specific regulatory modules, and are largely biased to regulatory modules near (within 100 kb) the transcription start site or within the known promoter regions.

Disclosed herein are methods and systems which utilize an unsupervised transfer learning paradigm to perform cross cell-type expression modeling. Supported by a representative and comprehensive unsupervised training dataset, a self-supervised transformer learns general regulatory mechanisms. Trained by a large number of cell types, the model can transfer the knowledge of regulatory mechanisms to produce expression prediction in unseen cell types. Finally, the disclosed model has a number of downstream applications including in silico perturbation of regulatory elements, non-coding mutation effect evaluation, enhancer-promoter target prediction, and transcription factor interaction analysis.

General expression transformer (GET) is an interpretable foundation model for transcription regulation across 213 human fetal and adult cell types that exhibits universal applicability and exceptional accuracy. GET learns transcriptional regulatory syntax from chromatin accessibility data across hundreds of diverse cell types and successfully predicts gene expression in both seen and unseen cell types, approaching experimental accuracy. The versatile nature of GET allows it to be transferred to different sequencing platforms and measurement techniques. Additionally, it offers zero-shot prediction of reporter assay readout in new cell types, potentiating itself as a prescreening tool for cell type specific regulatory elements. GET outperforms previous state of the art models in identifying cis-regulatory elements, and identifies novel and known upstream regulators of fetal hemoglobin. Through interpreting GET, rich regulatory insight was provided for almost every gene in 213 cell types. Using coregulation information predicted by GET, causal discovery was performed to pinpoint potential transcription factor-transcription factor interactions and constructed a structural interaction catalog of human transcription factors and coactivators. Using information provided by GET, a lymphocyte-specific transcription factor-transcription factor interaction involving PAX5 and retinoic acid receptor family transcription factors was successfully identified, and a possible disease driving mechanism of a lymphoma-associated germline-variants through affecting the binding of PAX5 disordered region to the nuclear receptor domain of retinoic acid receptors was highlighted. Overall, GET's broad applicability and profound understanding of transcription regulation can advance understanding of noncoding genetic variants and guide de novo design of cell-type specific transcriptional regulatory circuits and transcription factors for synthetic biology application.

GET may further incorporate of multiple layers of biological information, including but not limited to nucleotide-level ATAC footprints, three-dimensional chromatin architecture, and regulator expression profiles. Multiplexed nucleotide-level perturbations or randomizations can be used to calibrate GET for precise predictions of the functional impact of noncoding genetic variants. The evergrowing single-cell multi-omics datasets offer enormous potential for training GET on continuous cellular trajectories and perturbed states, thereby imparting the model with a dynamic understanding of cell state transitions. Leveraging GET as a computational framework, generative models can be developed to design megabase-scale enhancer arrays and engineer cell-type specific transcription factors or their interaction inhibitors for targeted therapeutic interventions. Collectively, GET represents an approach in cell type-specific transcriptional modeling, with broad applicability in the identification of regulatory elements, upstream regulators, and crucial transcription factor interactions.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “optional” or “optionally” means that the subsequent described event, circumstance, or substituent may or may not occur, and that the description encompasses instances where the event or circumstance occurs and instances where it does not.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

“Biological sample,” “sample,” “test sample,” “sample from a subject,” and “patient sample” as used interchangeably herein may be a sample of blood, such as whole blood (including for example, capillary blood, venous blood, dried blood spot, etc.), tissue, urine, serum, plasma, amniotic fluid, an anal sample (such as an anal swab specimen), lower respiratory specimens such as, but not limited to, sputum, endotracheal aspirate or bronchoalveolar lavage, nasal mucus, cerebrospinal fluid, placental cells or tissue, endothelial cells, leukocytes, or monocytes. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. A variety of cell types, tissue, or bodily fluid may be utilized to obtain a sample. Such cell types, tissues, and fluid may include sections of tissues such as biopsy and autopsy samples, oropharyngeal specimens, nasopharyngeal specimens, nasal mucus specimens, frozen sections taken for histologic purposes, blood (such as whole blood, dried blood spots, etc.), plasma, serum, red blood cells, platelets, an anal sample (such as an anal swab specimen), interstitial fluid, cerebrospinal fluid, etc. Cell types and tissues may also include lymph fluid, cerebrospinal fluid, or any fluid collected by aspiration. A tissue or cell type may be provided by removing a sample of cells from a human and a non-human animal, but can also be accomplished by using previously isolated cells (e.g., isolated by another person, at another time, and/or for another purpose). Archival tissues, such as those having treatment or outcome history, may also be used.

The term “gene” refers to a DNA sequence that comprises control and coding sequences necessary for the production of an RNA having a non-coding function (e.g., a ribosomal or transfer RNA), a polypeptide, or a precursor of any of the foregoing. The RNA or polypeptide can be encoded by a full length coding sequence or by any portion of the coding sequence so long as the desired activity or function is retained. Thus, a “gene” refers to a DNA or RNA, or portion thereof, that encodes a polypeptide or an RNA chain that has functional role to play in an organism.

The term “machine learning model” refers to a collection of parameters and functions, where the parameters are trained on a set of training samples. The parameters and functions may be a collection of linear algebra operations, non-linear algebra operations, and tensor algebra operations. The parameters and functions may include statistical functions, tests, and probability models. The model can learn from the training samples in a training process that optimizes the parameters to provide an optimal quality metric for classifying test samples. The training function can include expectation maximization, maximum likelihood, Bayesian parameter estimation methods such as markov chain monte carlo, gibbs sampling, hamiltonian monte carlo, and variational inference, or gradient based methods such as stochastic gradient descent, Adam optimization and the Broyden-Fletcher-Goldfarb-Shanno (BFGS) algorithm. Example parameters include weights (e.g., vector or matrix transformations) that multiply values, e.g., in regression or neural networks, families of probability distributions, or a loss, cost or objective function that assigns scores and guides model training. A model can include multiple submodels, which may be different layers of a model or independent model, which may have a different structural form, e.g., a combination of a neural network and a support vector machine (SVM). Examples of machine learning models include deep learning models (e.g., transformer), neural networks (e.g., deep learning neural networks), kernel-based regressions, adaptive basis regression or classification, Bayesian methods, ensemble methods, logistic regression and extensions, Gaussian processes, support vector machines (SVMs), a probabilistic model, and a probabilistic graphical model. A machine learning model can further include feature engineering (e.g., gathering of features into a data structure such as a 1, 2, or greater dimensional vector) and feature representation (e.g., processing of data structure of features into transformed features to use in training for inference of a classification).

A “subject” or “patient” may be human or non-human and may include, for example, animal strains or species used as “model systems” for research purposes, such a mouse models, prokaryotic models (e.g., bacteria), archea, and single-celled eukaryotes(e.g., yeast). Likewise, a subject may include either adults or juveniles (e.g., children). Moreover, patient may mean any living organism, preferably a mammal (e.g., humans and non-humans) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment, the mammal is a human.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Identifying Genetic Regulatory Modules

In some embodiments, provided herein are computer implemented methods for identifying genetic regulatory modules. Regulatory modules include those regions of the genome, near or far from the transcription start site, which effect transcription or expression of a gene. The methods comprise obtaining sequence features of genomic regions and expression data for one or more target genes from a biological sample or database; determining interaction between sequence features; generating a score representing the effect on expression of a target gene for the sequence features or combinations thereof; and identifying regulatory modules based on the score.

In some embodiments, the methods further comprising assigning a regulatory module as a transcriptional enhancer or transcriptional repressor based on the score. For example, scores indicative of regulatory modules which increase gene transcription may be considered transcriptional enhancer whereas those scores indicative of regulatory modules which decrease gene transcription may be considered a transcriptional repressor. The score may be compared to a scoring metric which provides values, averages, cut-offs for expression effects of enhancers and repressors. The score may be reported such that those regulatory modules which do not affect expression are given a score of zero, those which enhance expression are given a positive score, and those that repress expression have a negative value, with increasing positive and negative values indicating greater effect on the expression.

Sequence features include data regarding a plurality of genetic features such as, but not limited to, transcription start sites, transcription factor binding sites, chromatin accessibility, nucleosome positioning or occupancy, and the like. In some embodiments, the sequence features comprise chromatin accessibility data, transcription factor binding motif data, nucleotide sequences (e.g., nucleotide-level sequence data), or a combination thereof.

Chromatin accessibility is a measure of the accessible or non-protected areas of the genome. Thus, the input comprises regions of DNA which are accessible to transcription factors which promote gene expression. Generally, chromatin accessibility data is gathered by assays which separate the genome by enzymatic or chemical means and isolate either the accessible or protected locations. The isolated DNA is then quantified using a sequencing platform (e.g., next generation sequencing). Chromatin accessibility assays that directly isolate accessible locations of a genome include DNase I hypersensitive sites sequencing (DNase-seq; Song L, and Crawford G E. Cold Spring Harb Protoc. 2010 February; 2010(2):pdb.prot5384), Formaldehyde-Assisted Isolation of Regulatory Elements (FAIRE; Giresi P G, et al., Genome Res. 2007 June; 17(6):877-85), and Assay for Transposase-Accessible Chromatin (ATAC; Buenrostro J D, et al, Curr Protoc Mol Biol. 2015 Jan. 5; 109:21.29.1-21.29.9) whereas micrococcal nuclease digestion methods (e.g., MNase-seq; Mieczkowski, J., et al., Nat Commun 7, 11485 (2016)) indirectly evaluates chromatin accessibility. In some embodiments, the chromatin accessibility data comprises nucleotide level accessibility data. In certain embodiments, the chromatin accessibility data comprises Assay for Transposase-Accessible Chromatin (ATAC) data. In certain embodiments, the chromatin accessibility data is mined from available resources. In certain embodiments, the chromatin accessibility data is gathered using an assay described above.

In some embodiments, transcription factor binding motif data is compiled by scanning the chromatin accessibility data for regions which include sequences known for binding by transcription factors and the sequences themselves. In some embodiments, the predicted binding affinity of the transcription factor binding sites along with the region or sequence accessibility data is utilized to form the final input for the method.

In some embodiments, the expression data comprises proteomic (e.g. information regarding protein expression, posttranslational modifications, spatial configurations, intracellular localizations, interactions between proteins, and interactions between proteins and other molecules) or transcriptomic data (e.g., information regarding the quantity, structure, composition, and/or location of ribosomal RNA (rRNA), messenger RNA (mRNA), transfer RNA (tRNA), micro RNA (miRNA), and other non-coding RNA (ncRNA)). In select embodiments, the expression data comprises RNA-seq data (e.g., RNA-seq data, single cell RNA-seq data (scRNA-seq), nucleus RNA-seq data, or single nucleus RNA-seq data (snRNA-seq)). In certain embodiments, the expression data is gathered from pre-existing available resources. In certain embodiments, the expression data can be generated using single cell sequencing or single nuclei sequencing. In certain embodiments, the expression data is generated by conducting single cell RNA sequencing (see, e.g., Kalisky, T., Blainey, P. & Quake, S. R. Annual review of genetics 45, 431-445, (2011); Kalisky, T. & Quake, S. R. Nature Methods 8, 311-314 (2011); Islam, S. et al. Genome Research, (2011); Tang, F. et al. Nature Protocols 5, 516-535, (2010); Tang, F. et al. Nature Methods 6, 377-382, (2009); Ramskold, D. et al. Nature Biotechnology 30, 777-782, (2012); and Hashimshony, T., Wagner, F., Sher, N. & Yanai, I. CEL-Seq:. Cell Reports, Cell Reports, Volume 2, Issue 3, p 666-673, 2012). In certain embodiments, the expression data is generated by conducting nucleus RNA sequencing (see, e.g., Swiech et al., 2014, Nature Biotechnology Vol. 33, pp. 102-106; Habib et al., 2016, Science, Vol. 353, Issue 6302, pp. 925-928; Habib et al., 2017, Nat Methods. 2017 October; 14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., bioRxiv 74674).

The genomic regions from which the sequence features are obtained may be various lengths away from the transcription start site and may be 5′ or 3′ from the transcription start site. In some embodiments, the genomic regions comprise regions within 20 kilobases (kb) (e.g., within 10 kb, within 5 kb, within 1 kb) of the transcription start site of a target gene. In some embodiments, the genomic regions comprise regions within 2 Megabases (Mb) (e.g., within 1.5 Mb, within 1 Mb, within 0.75 mb, within 0.5 Mb, within 0.25 Mb, within 0.1 Mb, or within 50 kb) of the transcription start site of a target gene. In some embodiments, the genomic regions comprise regions greater than 1 Megabase (e.g., 1.2, 1.4, 1.5, 1.6. 1.8, or 1.0 Mb) from the transcription start site of a target gene. Thus, any number of genomic regions across an area within 2 Mb of the transcription start site of the one or more target genes can be used.

In certain embodiments, the sequence features and/or expression data are derived from a single cell. In some embodiments, the single cell is an adult cell. In some embodiments, the single cell is a fetal cell. In some embodiments, the cell is a human cell.

Accordingly, in some embodiments, the sequence data may comprise single cell ATAC-seq and the expression data may comprise single cell RNA-seq or single cell nucleus RNA-seq data. Alternatively, in certain embodiments, the sequence features and/or expression data are derived from a single cell type (e.g., cells from a specific anatomical region, tissue, course, or cell-line). In some embodiments, the sequence features and/or expression data features are derived from bulk cell data. The methods are not limited by the type of cells in which the data is derived. In some embodiments, the data may be from a specific subset of cells, e.g., distinct developmental stages, cells of a particular disease or class of disease (e.g., tumor or developmental disease), or normal cells of a particular type or class.

In some embodiments, a deep learning model is used to determine the interaction between sequence features and/or correlating the effect on expression of a target gene for the sequence features or combinations thereof. Deep learning architectures include, without limitation, deep neural networks, deep belief networks, deep reinforcement learning, recurrent neural networks, convolutional neural networks and transformers. In select embodiments, the deep learning model comprises a transformer model.

As used herein, the term “transformer model” refers to deep learning models with the core idea self-attention, the ability to attend to different positions of the input sequence to compute a representation of that sequence. A transformer model models sequential information (sequences) using stacked self- and cross-attention layers. In some embodiments, the transformer model uses an encoder-decoder architecture in which the encoder has encoding layers that process the input iteratively one layer after another, while the decoder has decoding layers that do the same with the output of the encoder. Transformer models include various language/foundation models, including, for example, BERT, RoBERTa, Xlnet, Albert, GPT, ViT etc.

### Predicting Target Gene Regulation and Expression

Also disclosed herein are computer implemented methods for predicting target gene regulation and expression. The methods comprise identifying regulatory modules for a target gene and generating a score representing the effect on target gene expression for one or more identified regulatory modules with a machine learning model. Identifying regulatory modules may be accomplished using a method as disclosed herein.

In some embodiments, the methods further comprise generating a score for one or more modified regulatory modules. Modified regulatory modules include any regulatory module which comprise a modification to one or more features (e.g., chromosome rearrangements) of the regulatory modules.

In some embodiments, the modification comprises a chromosome rearrangement (e.g., deletions, inversions, duplications, and translocations of the chromosome). A deletion means that part of a chromosome is missing. An inversion occurs when a piece of chromosome is flipped and then reattached in place, resulting in a different order to the genes and surrounding sequences. A duplication means that the chromosome has an extra segment of DNA repeated. A translocation occurs when one chromosome loses a piece of DNA which proceeds to join with a non-homologous chromosome.

In some embodiments, the modification comprises one or more nucleic acid substitutions, deletions, and/or insertions in a sequence of a regulatory module. For example, the modification may comprise a single substitution, a single deletion, or single insertions within the sequence of the regulatory module. Alternatively, the modification may comprise multiple substitutions, multiple deletions, multiple insertions, or a combinations of one or more of a substitution, insertion, and deletion. When the regulatory module is in a non-coding region of the genome, these modifications may be referred to as non-coding mutations.

The modified regulatory modules may be known to be associated with a change in gene expression. The modified regulatory modules may be known to be associated with a disease or disorder to which the effect on expression is unknown.

The methods disclosed herein can be used to determine the effect on expression of known mutations or chromosome rearrangements or may be used to engineer mutations or modifications in a regulatory modules to confer a desired effect on expression (e.g., decrease or increase gene expression). Accordingly, the methods disclosed herein may be used to design chromosomal modifications to correct a disease-causing modifications to gene regulatory modules or to design chromosomal modifications to confer a desired therapeutic effect on gene expression (e.g., to increase expression of gene product in a subject with a disease or disorder).

Thus, in some embodiments, the methods may further comprise designing, and optionally conducting, one or more gene editing experiments to generate a modified regulatory module. Gene editing experiments include any methods which can confer mutations to the genome of a cell, cell line, or subject. Non-limiting examples of gene editing experiments based upon technologies using clustered regularly interspaced short palindromic repeats (CRISPR)-CRISPR-associated protein (Cas), transcription activator-like effector nucleases (TALENs), zinc-finger nucleases (ZFNs), and homing endonucleases or meganucleases. In some embodiments, synthetic expression systems (e.g., genomes) are generated containing one or more modifications identified by the methods described herein. Such synthetic systems find use, for example, in research applications (e.g., drug screening applications).

In some embodiments, the methods comprise generating a score representing the effect on target gene expression for one or more identified regulatory modules with a machine learning model. Any appropriate machine learning model may be used as appropriate within the embodiments of the disclosed methods. In some embodiments, the machine learning model comprises a language model. As used herein, the term “language model” refers to a probability distribution over sequences of text or words.

The machine learning model may be trained using techniques such as unsupervised, supervised, self-supervised, semi-supervised, reinforcement learning, transfer learning, incremental learning, curriculum learning techniques, and/or learning to learn. In select embodiments, training for the machine learning model is selected from the group consisting of unsupervised learning, self-supervised learning, semi-supervised learning, transfer learning, and combinations thereof.

Unsupervised learning comprises methods in which deep learning techniques are used to find implicit patterns in the data without being explicitly trained on labeled data. Unlike supervised learning, unsupervised learning does not rely on labeling or annotation and a feedback loop. Semi-supervised learning is a machine learning method mix of supervised and unsupervised learning in which there is input data, and a fraction of the input data is labeled as the output. Self-supervised learning is a machine learning process where the model trains itself to learn one part of the input from another part of the input. It is also known as predictive or pretext learning. In this process, the unsupervised problem is transformed into a supervised problem by auto-generating the labels. Self-supervised learning and unsupervised learning methods can be considered complementary learning techniques as both do not need labeled datasets. Unsupervised learning can be considered as the superset of self-supervised learning as it does not have any feedback loops. On the contrary, self-supervised learning has a lot of supervisory signals that act as feedback in the training process. Transfer learning is the reuse of a pre-trained model on a new problem.

The machine learning model may be trained using input data obtained from biological samples. The machine learning model may be trained using input data obtained from publicly available sources, e.g., literature and database sources.

Training typically occurs after selection and development of a machine learning model and before the machine learning model is operably in use. However, the model may be refined with additional inputs over time as new data to train the model with is gathered. The training data used to teach the machine learning model can comprise input data such as multi-omic data. Multi-omic data may include proteomic, transcriptomic, genomic (e.g., single nucleotide variations, indels in tumor and normal, structural rearrangements, copy number variations, gene fusions, and expressed variants for tumor genomes), epigenetic, chromatin accessibility data, microbiomic, lipidomic, metabolomic, functional or phenotypic, imaging, and literature data.

In certain embodiments, the training data comprises transcriptomic data. In select embodiments, the training data comprises, consists of, or consists essentially of single cell RNA-seq data (scRNA-seq) or single nucleus RNA-seq data (snRNA-seq). In certain embodiments, the training data comprises, consists of, or consists essentially of chromatin accessibility data. In some embodiments, the chromatin accessibility data comprises nucleotide level accessibility data. In certain embodiments, the training data comprises, consists of, or consists essentially of ATAC-seq. In some embodiments, the training data comprises, consists of, or consists essentially of single cell data. In certain embodiments, the training data is multi-omic single cell data. In certain embodiments, the multi-omic data is transcriptomic data and chromatin accessibility data. In certain embodiments, the multi-omic data is single cell RNA-seq or single cell nucleus RNA-seq data and ATAC-seq.

The training may comprise, consist of, or consist essentially of obtaining sequence features of genomic regions and expression data for a plurality of target training genes from a single cell or cell line (e.g., fetal and/or adult human single cell data); determining interaction between sequence features; and identifying putative regulatory modules based on the effect on expression for the sequence features or combinations thereof.

Sequence features include data regarding a plurality of genetic features such as, but not limited to, transcription start sites, transcription factor binding sites, chromatin accessibility, nucleosome positioning or occupancy, and the like. In some embodiments, the sequence features comprise, consist of, or consist essentially of chromatin accessibility data, transcription factor binding motif data, or a combination thereof.

Chromatin accessibility is a measure of the accessible or non-protected areas of the genome. Thus, for training the input comprises regions of DNA which are accessible to transcription factors which promote gene expression. Generally, chromatin accessibility data is gathered by assays which separate the genome by enzymatic or chemical means and isolate either the accessible or protected locations. The isolated DNA is then quantified using a sequencing platform (e.g., next generation sequencing). Chromatin accessibility assays that directly isolate accessible locations of a genome include DNase I hypersensitive sites sequencing (DNase-seq), Formaldehyde-Assisted Isolation of Regulatory Elements (FAIRE) and Assay for Transposase-Accessible Chromatin (ATAC) whereas micrococcal nuclease digestion methods (e.g., MNase-seq) indirectly evaluates chromatin accessibility. In some embodiments, the chromatin accessibility data comprises nucleotide level accessibility data. In certain embodiments, the chromatin accessibility data comprises, consists of, or consists essentially of Assay for Transposase-Accessible Chromatin (ATAC) data. In certain embodiments, the chromatin accessibility data is mined from available resources. In certain embodiments, the chromatin accessibility data is gathered using an assay described above.

Transcription factor binding motif data is compiled by scanning the chromatin accessibility data for regions which include sequences known for binding by transcription factors and sequences themselves. In some embodiments, the predicted binding affinity of the transcription factor binding sites along with the regions accessibility data is utilized to form the final input for the training.

In some embodiments, the expression data comprises proteomic or transcriptomic data. In select embodiments, the expression data comprises, consists of, or consists essentially of RNA-seq data (RNA-seq) or nucleus RNA-seq data (nRNA-seq). In certain embodiments, the expression data is mined from available resources. In certain embodiments, the expression data can be generated using single cell sequencing or single nuclei sequencing. In certain embodiments, the expression data is generated by conducting single cell RNA sequencing (see, e.g., Kalisky, T., Blainey, P. & Quake, S. R. Annual review of genetics 45, 431-445, (2011); Kalisky, T. & Quake, S. R. Nature Methods 8, 311-314 (2011); Islam, S. et al. Genome Research, (2011); Tang, F. et al. Nature Protocols 5, 516-535, (2010); Tang, F. et al. Nature Methods 6, 377-382, (2009); Ramskold, D. et al. Nature Biotechnology 30, 777-782, (2012); and Hashimshony, T., Wagner, F., Sher, N. & Yanai, I. CEL-Seq:. Cell Reports, Cell Reports, Volume 2, Issue 3, p 666-673, 2012). In certain embodiments, the expression data is generated by conducting single nucleus RNA sequencing (see, e.g., Swiech et al., 2014, Nature Biotechnology Vol. 33, pp. 102-106; Habib et al., 2016, Science, Vol. 353, Issue 6302, pp. 925-928; Habib et al., 2017, Nat Methods. 2017 October; 14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., bioRxiv 74674).

The genomic regions from which the sequence features are obtained may be various lengths away from the transcription start site and may be 5′ or 3′ from the transcription start site. In some embodiments, the genomic regions comprise regions within 20 kilobases (kb) (e.g., within 10 kb, within 5 kb, within 1 kb) of the transcription start site of a target training gene. In some embodiments, the genomic regions comprise regions within 2 Megabases (Mb) (e.g., within 1.5 Mb, within 1 Mb, within 0.75 mb, within 0.5 Mb, within 0.25 Mb, within 0.1 Mb, or within 50 kb) of the transcription start site of a target training gene. In some embodiments, the genomic regions comprise regions greater than 1 Megabase (e.g., 1.2, 1.4, 1.5, 1.6. 1.8, or 1.0 Mb) from the transcription start site of a target gene. Thus, any number of genomic regions across an area within 2 Mb of the transcription start site of the target training gene can be used.

In some embodiments, a deep learning model is used to determine the interaction between sequence features and/or correlating the effect on expression of a target gene for the sequence features or combinations thereof. Deep learning architectures include, without limitation, deep neural networks, deep belief networks, deep reinforcement learning, recurrent neural networks, convolutional neural networks, and transformers. In select embodiments, the deep learning model comprises, consists of, or consists essentially of a transformer model.

In some embodiments, the target gene is from the same cell or cell-type as the single cell or cell line used in the training. In some embodiments, the target gene is from a different cell or cell-type as the single cell or cell line used in the training, such that the methods facilitate cross cell-type expression prediction. Different cells may be cells of a different type (e.g., cells from a specific anatomical region, tissue, course, or cell-line), cells from a different organism, or different cell type from a different organism.

The methods described herein can be implemented in digital electronic circuitry, or in computer software, firmware, or hardware. The methods can be implemented as one or more computer programs, e.g., one or more modules of computer program instructions, encoded on computer storage medium for execution by, or to control the operation of, data processing apparatus. Alternatively, or in addition, the program instructions can be encoded on an artificially generated, propagated signal, for example, a machine-generated electrical, optical, or electromagnetic signal that is generated to encode information for transmission to suitable receiver apparatus for execution by a data processing apparatus. In some embodiments, the methods are implemented as a non-transitory computer-readable medium storing instructions executable by one or more processors to perform operations.

The present disclosure also provides non-transitory computer-readable media. The non-transitory computer-readable media stores instructions that when executed by one or more processors performs some or all of the operations described in the disclosed methods.

In some embodiments, the one or more processors perform operations comprising at least one or all of: obtaining sequence features of genomic regions and expression data for one or more target genes from a biological sample or database; determining interaction between sequence features; generating a score representing the effect on expression of a target gene for the sequence features or combinations thereof; identifying regulatory modules based on the score; generating modifications of a regulatory module, and determining and/or reporting a score for a modified regulatory module.

The methods described herein can be implemented as a system including one or more processors and a computer-readable medium storing instructions executable by the one or more processors to perform operations, as described above. The system may comprise at least one computer system comprising the one or more processors and/or the computer-readable media. The system may further comprise one or more local servers or databases connected to or integrated with the one or more computer systems. The system may further comprise one or more devices integrated with the computer systems.

In some embodiments, the system generates and displays or otherwise reports results to a user and/or feeds results to an instrument for downstream use. For example, in some embodiments, the system displays a sequences, graphs, probabilities, or other results to a user. For example, the result may include one or more sequences that are predicted to elicit a desired change in expression of one or more genes. The result may also include the effect of expression of the new or modified sequences on genes that were not the original target gene for which the prediction was being completed.

The nature of the data reported to the user may be based on a parameters previously selected by the user (e.g., using a user interface) including for example certain levels or percentages of change in expression of the one or more genes. In some embodiments, the data is fed to an instrument, such as a nucleic acid synthesizer, protein/peptide synthesizer, cell culture instrument, gene modification instrument, or other device. In response to the data, the instrument synthesizes, modifies, or alters a molecule (e.g., nucleic acid molecule, peptide, etc.), cell, tissue, organism, etc. For example, in some embodiments, the instrument synthesizes a modified nucleic acid regulatory sequence that finds use in the genetic modification of a DNA molecule, a cell, a tissue, or an organism. For example, in some embodiments, the instrument designs and/or synthesizes modified genetic engineering machinery, or nucleic acids encoding thereof, for use in modifying a regulatory module sequence at a desired location within a genome of an organism (e.g., designs and/or synthesizes guideRNA for use with CRISPR/Cas methods, designs and/or synthesizes modified nucleases (e.g., Cas nucleases, TALENs, ZFNs, and homing endonucleases or meganucleases) to recognize sequences surrounding the site of the modified nucleic acid regulatory module).

### EXAMPLES

The following are examples of the present invention and are not to be construed as limiting.

### Example 1

Unseen cell-type gene expression modeling aims to predict gene expression of cell-types that is not listed among the training cell types using cell-type specific genomic or epigenomic features. Given a training set of input genomic regions G with various sequence, motif or accessibility features and target gene expression Yin those regions, the training data distribution from a particular cell type i is denoted as a joint distribution Pᵢ(Gᵢ, Yᵢ) over Gᵢ×Yᵢ. Similarly, the test data distribution is defined as a joint distribution P′(G′, Y′) over the test set G′×Y′. When P(G, Y) and P′(G′, Y′) are drawn from different cell types, P′(G′, Y′) is different from P(G, Y). In practical situation, Y′ could be unavailable. Since P and P′ (regulatory mechanisms) show cell-type specificity while at the same time governed by cell-type-agnostic factors (genomic DNA, protein-protein interaction, etc.), it's possible to first learn from a large amount of examples Pᵢ(Gᵢ, Yᵢ) to model the entire P distribution family (or functional), such that one can make prediction of Y′ using G′, even if they P′(G′,Y′) are not sampled in the training data.

In order to manage this, an unsupervised transfer learning paradigm was leveraged with a self-supervised transformer named gene expression transformer (GET) by transferring general representations of regulatory elements and transcription factors to unseen cell type gene expression prediction. GET includes the following two stages.
Unsupervised accessible genome modeling that: sequence features of cell-type specific accessible regulatory regions are taken as input and a self-supervised transformer is adopted to learn general cis- and trans-interactions between regulatory elements and potential binding transcription factors from various tissue or cell types.
Transfer learning to predict: The trained model is fine-tuned in the first stage on the OOD training set of input G and target Y and then the model is tested on the testing set G′,Y′ with non-overlapping cells with respect to training data.

Prior to the first stage, a curated training dataset with paired RNA-seq and ATAC-seq data is processed into hundreds of thousands of training samples that cover ˜2-4 Megabase of genomes, each with paired sequence feature, accessibility feature and expression label. Transcription initiation involves the binding of transcription factors and polymerase to regulatory elements or promoters, which typically need the DNA to be partly dissociated from histone protein and become accessible. Thus, chromatin accessibility can be used as a coarse cell-type specific filter on the genome to select regions that are relevant to transcription regulation in a particular cell type.

While previous studies have used tissue-specific epigenomic/transcriptomic datasets from large consortia including ENCODE (ENCODE Project Consortium et al. Nature, 489(7414):57, 2012), ROADMAP (Kundaje, et al., Nature, 518(7539):317-330, February 2015), and FANTOM5 (Noguchi, et al., Scientific Data, 4(1):170112, August 2017), the cell-type coverage of one single assay like ATAC-seq is often limited and incomprehensive. The curated dataset though comes from two comprehensive single-cell ATAC-seq atlas (Domcke, et al., Science, 370(6518):eaba7612, 2020; Kai, et al., Cell, 184(24):5985-6001.e19, 2021) that contains 222 human cell types across many tissues and fetal and adult stage, which comprehensively reflect the most accessible regions on the human genome in different cell types. This property supports the approach of using a self-supervised model to learn general regulatory mechanisms that are adapted to unseen cell types.

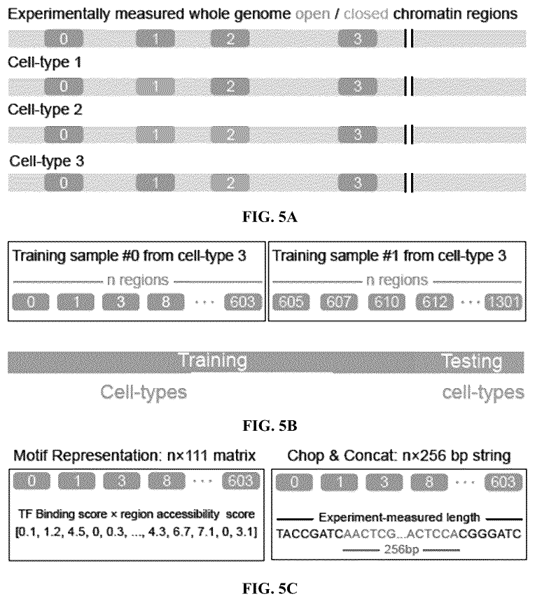

For finetuning the model, paired accessibility and expression information in the fetal cell types was used. Specifically, accessible regions are aggregated as a unified region pool, totaling 1,000,029 unique regions (with an average length of 506 bp). Then different cell types are sampled from the region pool, as shown in FIG. 5A. With open regions in different cell types as different instances, the dataset contains over 42 million (42,702,271) regions in total. Expression is usually determined by regulatory regions in proximity to gene promoters. Motivated by this insight, n consecutive regions are group into one sample, each covering around 2-4 Megabases of human genome. For the expression prediction task, the leave-one-out cell is used as the testing sample and all other cells as the training sample, as shown in FIG. 5B. For modeling the sequence pattern in each region, the transcription factor motif scanning result of open regions is used to summarize each region as a motif binding vector g∈R^{t}, where t=111 in the earlier version of GET, and 282, in the current version of GET. Each dimension in the vector corresponds to the predicted binding affinity of a different class of transcription factor binding sites. Further, the vector is multiplied or concatenated with the region's accessibility counts to form the final input, as illustrated in FIG. 5C. The correlation map of gene expression across 12 different cell types is shown in FIG. 5D.

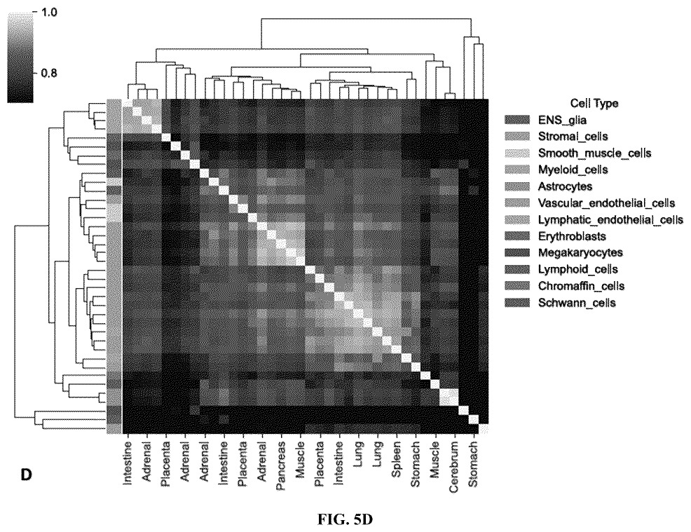

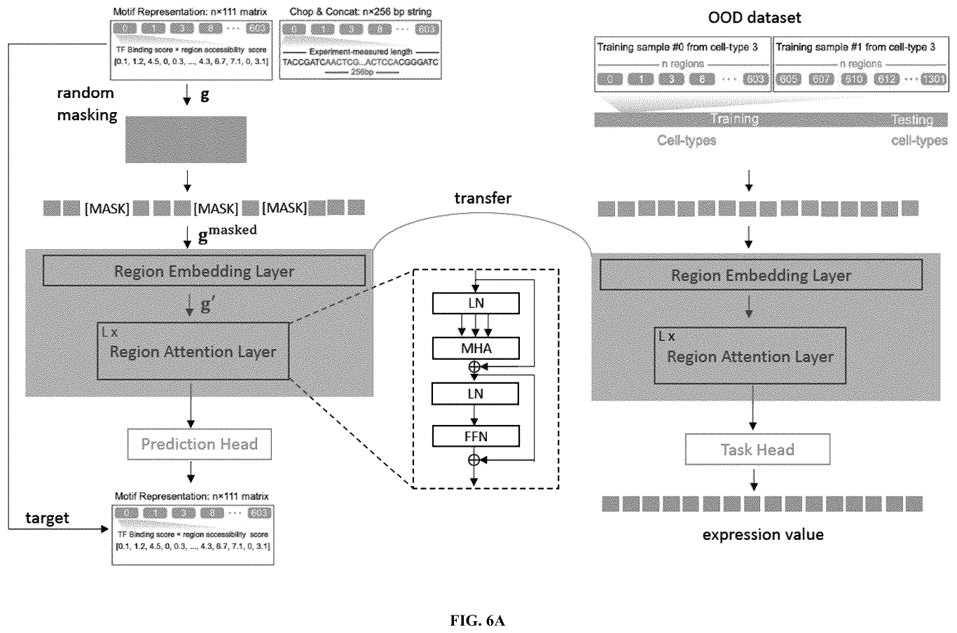

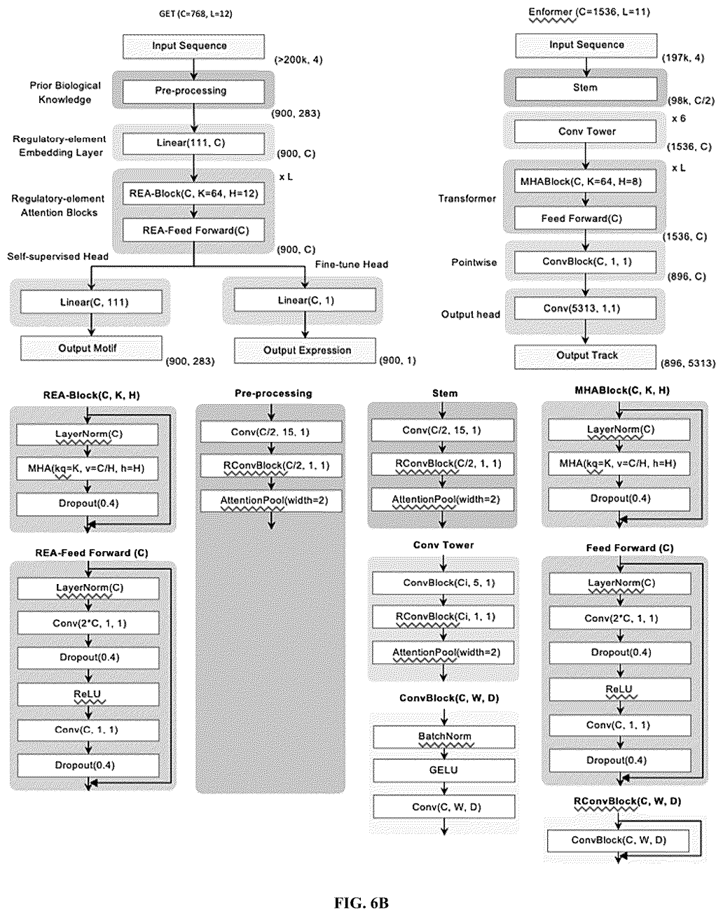

The unsupervised genome region transformer referred to GET for learning genome region representations from the proposed multi-organ chromatin accessibility-expression dataset is detailed, as shown in FIG. 6. First, n regions are taken as a sample for the input and is denoted as

$$g = {\{ {g}_{i} \}}_{i = 1}^{n} , g ∈ {R}^{n \times t} ,$$

where gᵢ is the vector of the i-th region obtained by the transcription factor motif scanning result. Then g is fed into one projection layer to generate the embedding of each region. Finally, multiple region-wise attention layers with Gaussian Error Linear Unit (GELU) (Hendrycks and Gimple arXiv:1606.08415, 2016) are applied to capture the general cis- and trans-interactions between regulatory elements and transcription factors.

In order to capture the general cis- and trans-interactions between regulatory elements and transcription factors from all kinds of human cell types in the MOC-AE dataset, a masked region modeling mechanism on the input sample g is adopted. Specifically, a random set of positions is uniformly selected to mask out

$$M = {\{ {m}_{i} \}}_{i = 1}^{k}$$

with a mask ratio of r=k/n. The regions in the selected positions are replaced with a [MASK] region. Thus, the masked input region is denoted as g^{masked}, to predict the original values of the masked regions M.

Masked regions embeddings of g^{masked} are taken as input to GET, while a simple linear layer is appended as the prediction head. Therefore, the overall objective of self-supervised training is formulated as equation 1:

$$L = E ( {\sum }_{i ∈ M} - log p ( {g}_{i} \vert {g}^{masked} ) )$$

where gᵢ denotes the i-th masked region to be predicted.

In this way, the learning paradigm is cell-type agnostic, that is, the model captures the commonality across cell types to make inferences on unseen cell types. Such a paradigm is reasonable, as the functioning of transcription machinery is dominated by cell-type agnostic biochemical properties. Such a paradigm is also feasible since the modeling of expression has a natural correspondence with existing natural language processing (NLP) settings. The expression process can be defined as an encoder-decoder system: a number of accessible regions determined are transformed by a “general regulatory mechanism” encoder, and the output embedding is decoded by RNA Polymerase II to a real number expression value. The entire input space is restricted by the whole human genome, where different cell types have different but overlapping subspaces as input. The output space is governed by the biochemical properties of the transcription machinery, which is mostly shared by different cell types.

After the self-supervised training on the MOC-AE dataset in the first stage, Gene-Former is transferred to the OOD gene expression prediction task. Specifically, the prediction head is replaced with a task head for the trained encoder and the model is fine-tuned end-to-end on the OOD training set of input G and target Y. For stabilized training, the input G is normalized with a min-max function and a log operator log (Y+1) is applied on the target Y. Following accepted practices, the model is trained to minimize a Poisson log-likelihood between the prediction Ŷ and the normalized target \tilde{Y}, which is formulated as equation 2:

$$\tilde{Y} ∼ Poisson ( \hat{Y} ) , \tilde{Y} = log ( Y + 1 )$$

Therefore, the objective of OOD transfer is defined as equation 3:

$${L}_{transfer =} \hat{Y} - \tilde{Y} \cdot log ( \hat{Y} )$$

where \tilde{Y}, Ŷ denote the normalized target and prediction of the model, separately. One cell type was left out as the test dataset while training on all other cell types. Then the model is tested on the testing set G′,Y′ with non-overlapping cells with respect to training data G,Y.

The method was evaluated on the OOD Gene Expression Dataset. Expression data was collected for different cell types across 15 fetal human organs. Expression values were represented as normalized transcript per million (TPM). The cell types were matched between RNA and ATAC datasets by annotating cell type names and ignoring those that cannot be matched. In total, 134 cell types had both accessibility and expression measurement. To improve training stability, the expression values were log-transformed as log₁₀(TPM+1). The gene expression was mapped to accessible regions using the following approach: if a region overlaps with a gene's transcription start site (TSS), the gene's expression value is assigned to that region as a label; if a region overlaps with multiple gene TSS, the expression values of the corresponding gene are summed up and used as the label of that region; if a region does not overlap with any TSS, the corresponding expression label is set as 0. Eventually, all regions are assigned as an expression label.

The following metrics were used to measure the difference between gene expression predictions and observations.

$$R 2 score {R}^{2} = 1 - \frac{{‖ Y - F ( G ) ‖}_{2}}{{‖ Y - Y ‖}_{2}} ; \qquad 1 )$$

$$Pearson R {r}_{p} = {p}_{F ( G ) , Y} = \frac{Cov ( F ( G ) , Y )}{{σ}_{F ( G )} {σ}_{Y}} ; \qquad 2 )$$

$$Spearman R {r}_{8} = {p}_{R ( F ( G ) ) , R ( Y )} = \frac{Cov ( R ( F ( G ) ) , R ( Y ) )}{{σ}_{R ( F ( G ) )} {σ}_{R ( Y )}} , \qquad 3 )$$

where F(⋅) represents the model. ∥⋅∥₂ is the Euclidean norm. G denotes the model input and Y denotes the target, which is the expression value. Cov(⋅) is the covariance matrix, and σ is the standard deviation. R(⋅) is the ranking function.

The implementation is based on PyTorch (Paszke, et al., Proceedings of Advances in Neural Information Processing Systems, 2019) framework. For self-supervised training, AdamW (Loshchilov and Hutter, International Conference on Learning Representations, 2019) was applied as the optimizer with a weight decay of 0.05 and a batch size of 256. The model is trained for 800 epochs with 40 warmup epochs for linear learning rate scaling. The maximum learning rate is set to 1.5e-4. The number Nₕ of multi-attention heads in the Region-wise Attention layer is set to 12, and the depth L of GET is 12. The embedding dimension D=768, and the default number of input regions n is set to 100. The default mask ratio is 0.25. For OOD transfer learning, AdamW is used as the optimizer with a weight decay of 0.05 and a batch size of 256. The model is fine-tuned for 100 epochs. For a comprehensive evaluation, 3 different random seeds are used for initialization to report the averaged results.

In the reset of Example 1, benchmark results for the earlier version of GET are reported. For a fair comparison with current state-of-the-art methods on unseen cell-type gene expression prediction, a similar training scheme for both Enformer and GET model were used. For a comprehensive comparison, three baselines were adopted, including 1) Enformer: which relies on DNA sequence as input; 2) GET (w/o stage 1): a vanilla transformer with random initialization; 3) GET (w/o stage 2): a pre-trained transformer without fine-tuning. The experimental results are reported in Table 1. The expression prediction performance was measured across all accessible regions and all gene transcription start sites (TSS).

TABLE 1

| | | Pearson | Spearman | | TSS | TSS | Avg |
|---|---|---|---|---|---|---|---|
| Model | R2 | R | R | TSS R2 | Pearson R | Spearman R | (↑) |
| Enformer | 27.1 | 85.8 | 28.7 | 39.2 | 64.1 | 65.3 | 51.7 |
| GET (w/o | 37.2 | 61.1 | 25.0 | 26.9 | 58.1 | 47.5 | 42.6 |
| stage 1) | | | | | | | |
| GET (w/o | 41.4 | 64.7 | 26.2 | 2.8 | 1.5 | 3.7 | 23.4 |
| stage 2) | | | | | | | |
| GET | 72.8 | 85.4 | 30.8 | 39.7 | 67.4 | 66.9 | 60.5 |
| GET (n = | 75.6 | 87.1 | 30.3 | 39.8 | 68.2 | 67.0 | 61.3 |
| 900) | | | | | | | |

GET with two stages achieves better performance than baselines in terms of all metrics. Specifically, when evaluated on the R2 score, the method outperforms Enformer, by a large margin, verifying the effectiveness in OOD gene expression prediction. Training from scratch without unsupervised genome region learning deteriorates the performance of GET. Furthermore, GET without the second stage achieves better performance than a vanilla transformer with random initialization. This validates the importance of self-supervised training in learning general cis- and trans-interactions between regulatory elements and transcription factors from all organs. Finally, adding the second stage to GET improves results with a clear growth, which implies that transfer learning is advantageous to unseen cell type gene expression prediction. The prediction result for all TSS was visualized with scatter plots in FIG. 7. (The performance of the current version of GET is shown along with it). Consistent with the correlation measure, GET shows significantly better agreement between predicted and observed expression value than other baselines. Furthermore, FIG. 8 shows the promising performance of unseen cell type gene expression prediction on 12 different leave-one-out cells (The performance of the current version of GET on cross-stage unsupervised prediction is shown along with it). These results indicate the strong generalizability of GET to other unseen cell types across life stages in the human body.

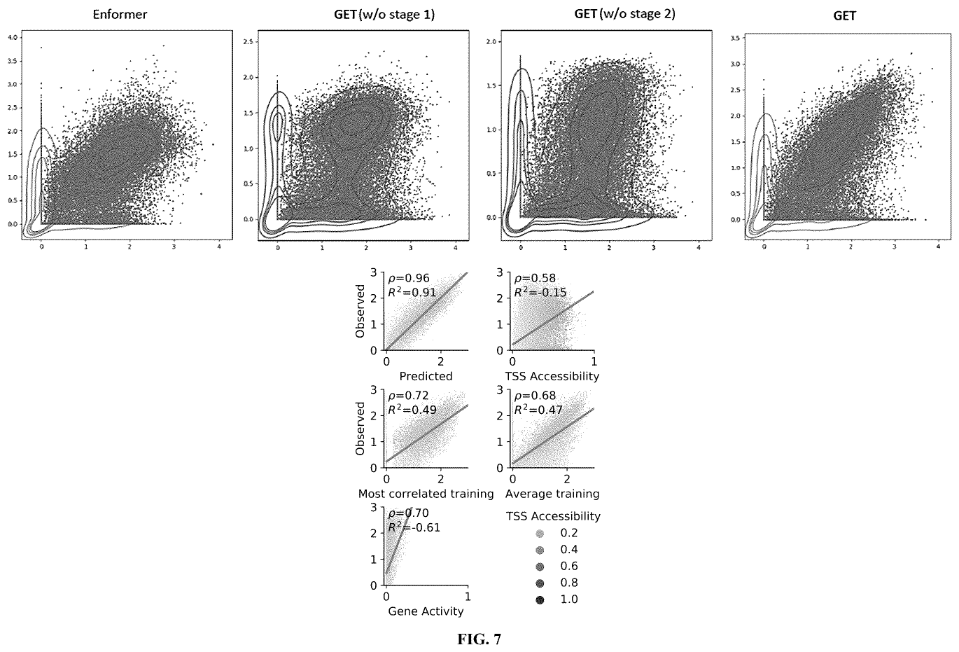

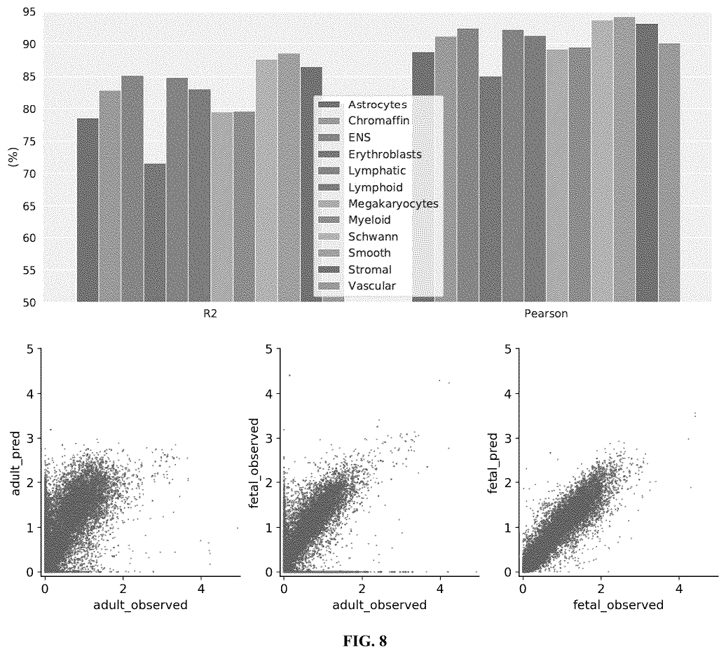

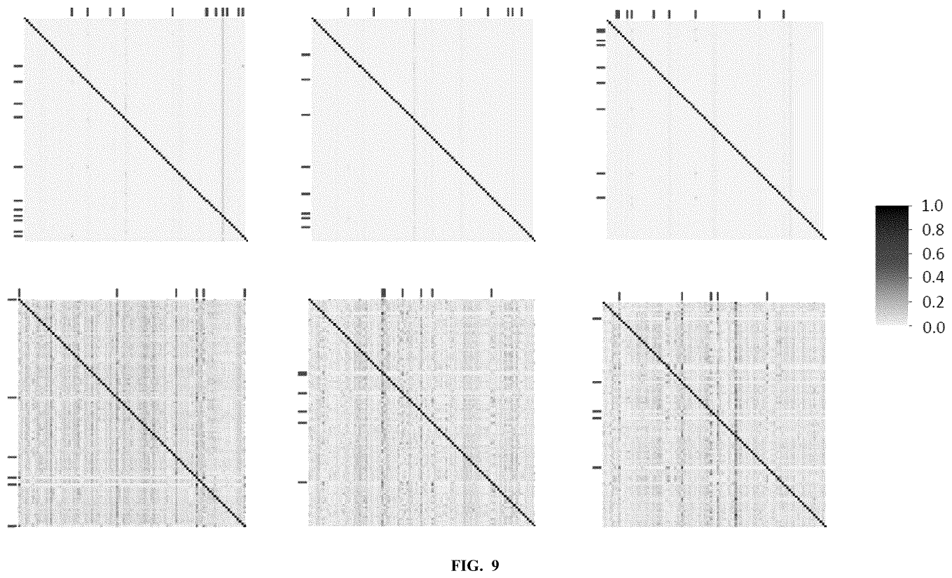

In order to quantify how the regulatory gene region information flow in GET, the information flow to input regions was approximated given attention weights. Specifically, three samples were randomly selected from the test set in the OOD Gene Expression Dataset and visualized attention maps of 100 regions in stage 1 (Top) and stage 2 (Bottom), as shown in FIG. 9. GET successfully aligned attention between the same regions and interactions between different regions in stage 1, thus demonstrating effectiveness in transmitting gene region information among region-wise attention layers. Meanwhile, transferred attention maps show vertical patterns that correspond to expressed TSSs (marked with red rectangles) and horizontal patterns on some regulatory regions, denoting active enhancers that regulate the corresponding gene and indicating the model can be used to generate biological hypotheses for downstream validation. Additional exemplary applications of GET are discussed in Example 2.

This model was also used for cell-type specific gene expression prediction in comparison to Enformer, Basenji2 (Kelley D R, et al., Genome Res. 2018 May; 28(5):739-750), Vaishnav et al., (Nature. 2022 March; 603(7901):455-463), ABC score (Fulco C P, et al., Nat Genet. 2019 December; 51(12):1664-1669), and gene activity (Granja J M, et al., Nat Genet. 2021 March; 53(3):403-411, and others). As shown in Table 2, the reported expression prediction accuracy (Spearman correlation) of each of the comparison is less than that of biological replications (˜0.94) but using the disclosed methods and models a Spearman correlation of ˜0.95 is achieved.

TABLE 2

| | | | Reported expression |
|---|---|---|---|
| | | Unseen | prediction accuracy |
| | | cell | (Spearman correlation, |
| Model | Organism | types? | Biological replicates ~0.94) |
| Herein | Human | Yes | ~0.95 |
| Enformer | Human/Mouse | No | ~0.85 |
| Basenji2 | Human/Mouse | No | ~0.81 |
| Vaishnav et al. | Yeast | — | ~0.9 |
| ABC Score | — | Yes | NA |
| Gene activity | — | Yes | ~0.3-0.5 |

### Example 2

A model as disclosed herein, in particular the resulting gradient score (hereinafter “Grad”), was compared with other models for identifying regions which effect gene expression, Enformer, DeepSEA (Zhou and Troyanskaya Nat Methods. October 2015; 12(10): 931-934), Genomic Evolutionary Rate Profiling (GERP; Cooper et al., Genome Res. 2005. 15: 901-913 and Davydov et al., PLoS Computational Biology 6 (12): e1001025), and a random baseline using base editing data in a fetal erythroblast cell line HUDEP. The results showed that Grad performed comparably to Enformer for regions close (<20 kbp) to the transcription start site (TSS), and outperformed Enformer for regions far (>20 kbp) from TSS. Additionally, Grad performed better than DeepSEA for all regions. These results demonstrate the potential for Grad in accurately predicting base editing sites in HUDEP cells, especially for regions far from TSS, as the model disclosed herein identified proximal regulatory elements as well as existing methods and distal regulatory elements better than existing methods in a cell type specific context. For the prediction using the model as disclosed herein fetal erythroblast data was used. For Enformer, pre-trained tracks related to erythroblast were used. DeepSEA and GERP are not cell-type specific and did not predict expression.

This model (GET) was also used for evaluating single nucleotide mutational effect on BCL11A expression and benchmarked with the same set of base editing data in HUDEP cell line against Enformer, DeepSEA, CADD (Rentzsch et al., Nucleic Acids Res. 2018 Oct. 29) and GERP. The result showed that GET outperforms most methods in distal mutation (>10kb), except for Enformer_abs, which does not produce directional (up/down-regulate) prediction result. For mutation <10kb within TSS, GET beats or comparable to Enformer, DeepSEA, and CADD. GERP is a conservation-based method that can be easily combined with GET to boost GET's performance. Note that in GET_ood, the model did not see Erythroblast expression in the training stage. The comparable performance of GET_ood to GET support a strong usecase of the method where the user can perform in-silico base editing for virtually any cell types with ATAC-seq information.

### Example 3

### Get, a Foundation Model for Transcription Regulation Across 213 Human Cell Types

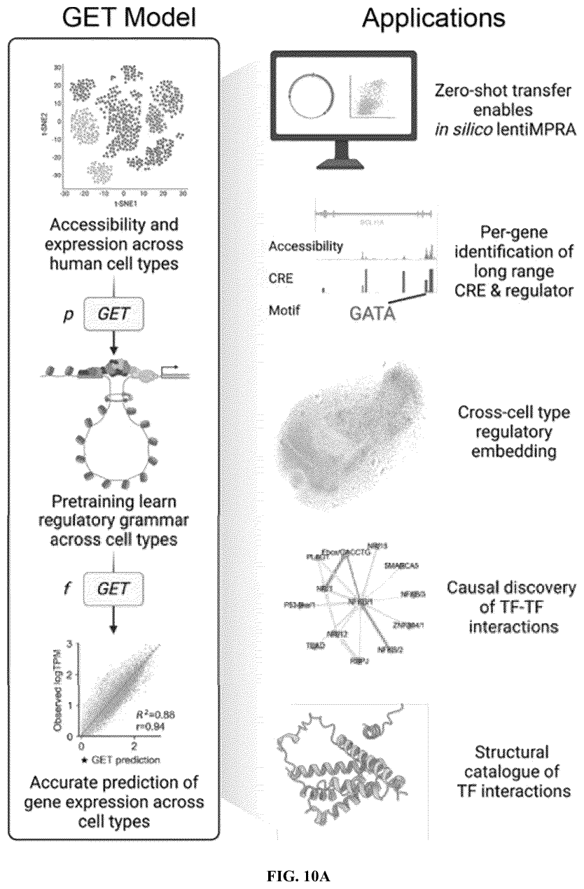

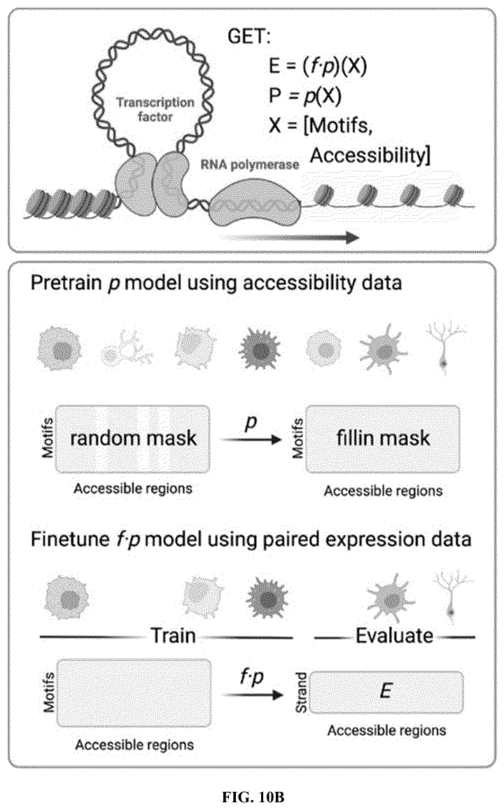

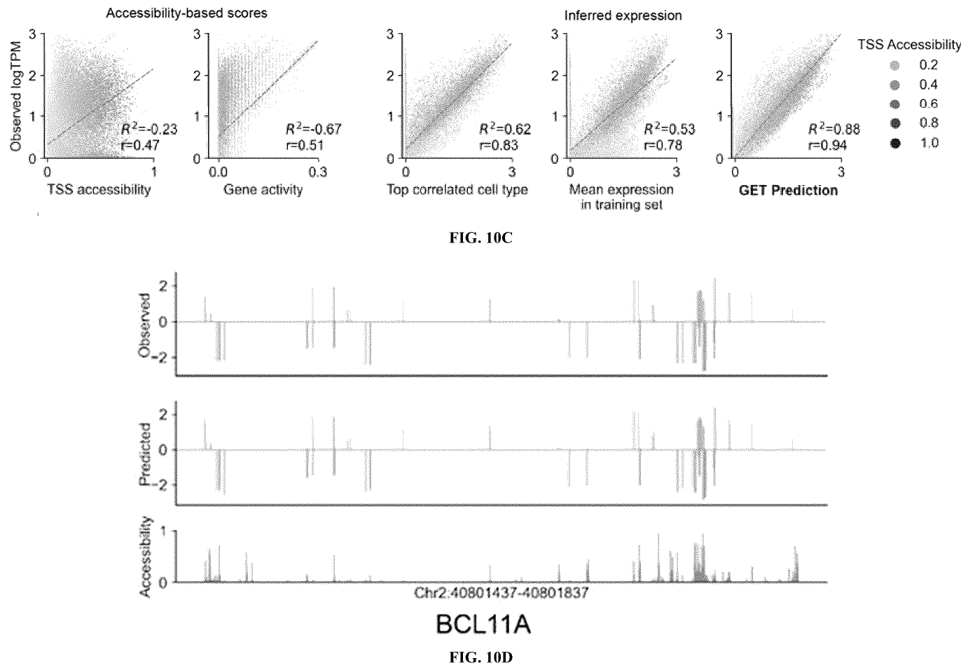

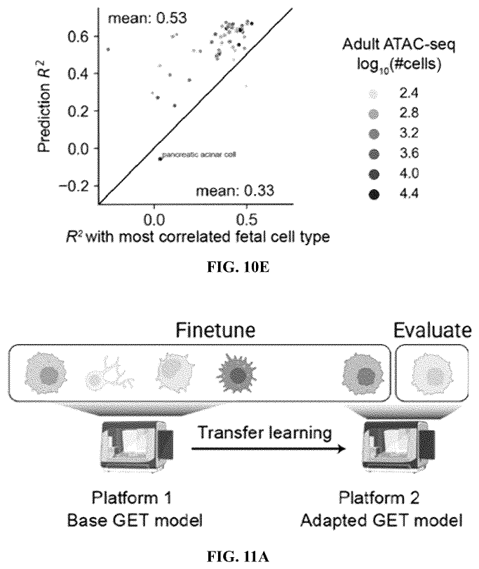

GET was developed as a novel foundational model to comprehend the transcription regulation across a diverse range of cell types. Unlike previous models such as Enformer, GET employs an extensive effective sequence length exceeding 2 Mbps and is not constrained to making predictions in only training cell types. The design philosophy of GET is rooted in the conceptual understanding of transcription regulation (FIG. 10B). At the forefront, Promoter and related contextual regulatory elements can be characterized by how well they bind different transcription factors (motif binding score, Methods) and how accessible they are in specific cell types. These features shape a chromatin environment (p(X)) that governs RNA polymerase II (PolII) functioning. Using an embedding and attention architecture specifically designed for the regulatory elements (Methods), self-supervised pretraining was performed to let GET learn how the regions and features interact with each other across diverse cell types. Specifically, by randomly masking out regulatory elements, the model is trained to predict the motif binding scores and optionally accessibility score in the masked region. Subsequently, PolII will translate the chromatin environment p(X) into an expression readout E (FIG. 10B). A finetuning stage with the same architecture but a different output head will simulate this process. This two-stage design makes it possible to use chromatin accessibility data with no paired expression measurement, greatly improving the diversity of regulation information in the training data.

The pretraining of GET uses of pseudobulk chromatin accessibility gathered from single cell assay for transposase-accessible chromatin with sequencing (scATAC-seq) data across 213 human fetal and adult cell types. Out of these, 153 were coupled with expression data, acquired either through a multiome protocol or separate single cell RNA sequencing (scRNA-seq) experiments. The motif binding score was calculated using known position weighted matrix and summarized according to sequence similarity to reduce feature redundancy. Assuming additivity in motif binding score, every sample is a region*feature matrices derived from a continuous range on the accessible genome across different cell types. This design of model input ensures both cell type specificity and generalizability while enabling efficient computational modeling. Strand-specific expression values are assigned to each region based on their overlap with expressed gene's promoters.

### Example 4

### GET Accurately Predicts Gene Expression in Unseen Cell Types at Experimental Accuracy

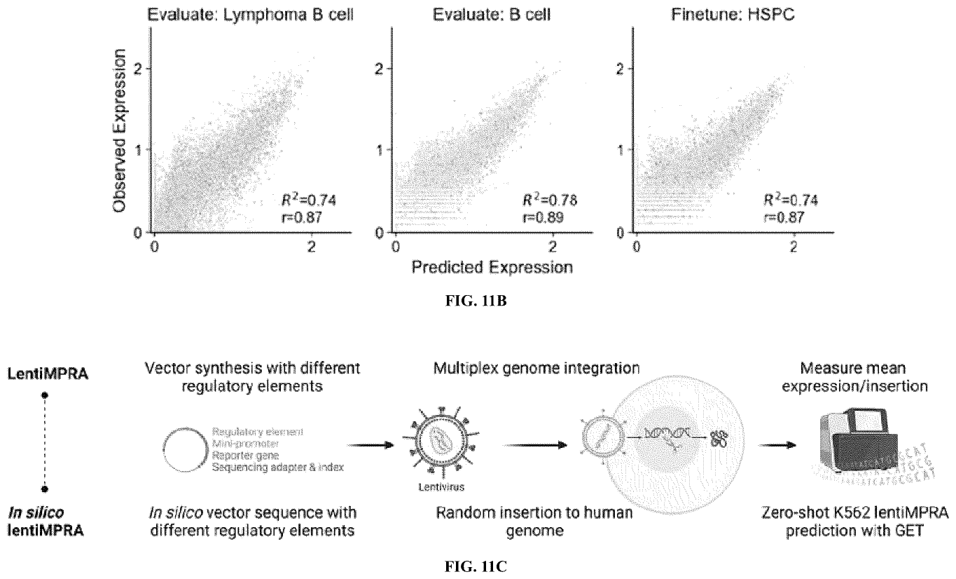

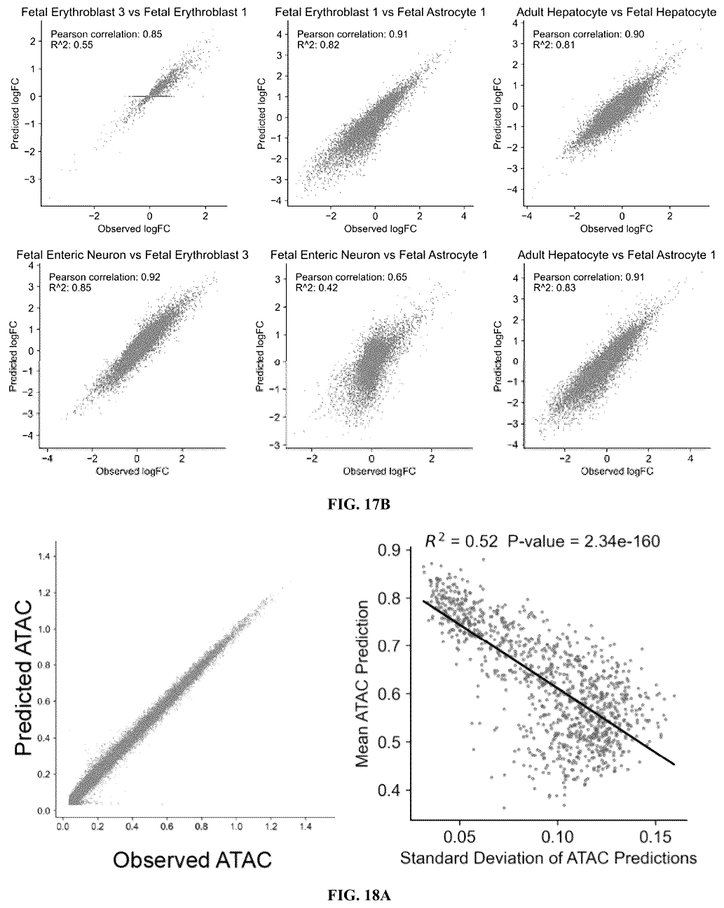

GET's ability to accurately predict gene expression in unseen cell types in a setting where one cell type is left out during the expression finetuning process was assessed. Remarkably, GET demonstrated the capacity to consistently predict the expression of the left-out cell types at a level of accuracy comparable to experimental standards, even when trained without quantitative accessibility signals. An example can be taken from left-out astrocytes, where the Pearson correlation between GET's predicted expression values and the observed expression reached 0.94 (R²=0.88), a result that is in line with experimental accuracy (Pearson r=0.92-0.99, FIG. 17A). GET's performance surpasses both transcription start site (TSS) accessibility (r=0.47, R²=−0.23) and gene activity score (r=0.51, R²=−0.67), emphasizing the significance of DNA sequence specificity and distal context information in transcription regulation. Furthermore, GET managed to outperform two robust benchmarks, including top correlated cell type expression (r=0.83, R² 0.62) and mean expression across training cell types (r=0.78, R²=0.53; as illustrated in FIG. 11). Additional validation was carried out, confirming GET's capability to make cell-type-specific predictions, as evidenced by a Pearson correlation of 0.91 (R²=0.82) between predicted and observed log fold change for Fetal astrocyte and Fetal erythroblast expression (see FIG. 17B).

GET's generalizability to adult cell types when trained solely on fetal data was also investigated. The findings showed an average R² of 0.53 across diverse adult cell types, once again surpassing the baseline (R²=0.33) obtained using corresponding fetal cell types for prediction (e.g., utilizing Fetal astrocyte to predict Adult astrocyte). The only 3 cell types where it cannot beat the baseline are cell types with low cell counts in either ATAC-seq or RNA-seq label (pancreatic acinar cell). This result reinforces the proposition that GET can extract common regulatory mechanisms that span across various cell types and stages of life.

To ascertain the impact of cross-cell-type pretraining on prediction performance, a GET model was finetuned from random initialization, which exhibited a substantial drop in performance compared to the pretrained version with the same number of training epochs (Pearson r: 0.596; Spearman rho: 0.642). Extending the training period for this baseline failed to enhance its performance (Pearson r: 0.607; Spearman rho: 0.658), highlighting the essential role of pretraining in facilitating model generalization.

In summation, by leveraging widely accessible ATAC-seq data and established transcription factor binding motifs GET acquires a broad understanding of the regulatory code, empowering it to predict unseen cell type expression with experimental precision.

### Example 5

### GET can be Transferred to Different Sequencing Platforms and Measurements

Diverse data generation platforms and processing methods often present a significant challenge for the universal generalizability of pretrained models. To assess whether GET can be transferred to a wholly new sequencing platform in such scenarios, its performance was benchmarked on 10× multiome sequencing of the lymph node (10×genomics.com/resources/datasets/fresh-frozen-lymph-node-with-b-cell-lymph oma-14-k-sorted-nuclei-1-standard-2-0-0; FIG. 11A, Methods) using the leave-cell-type-out evaluation approach. Notably, GET maintained consistent prediction outcomes for both finetuned or left-out cell types.

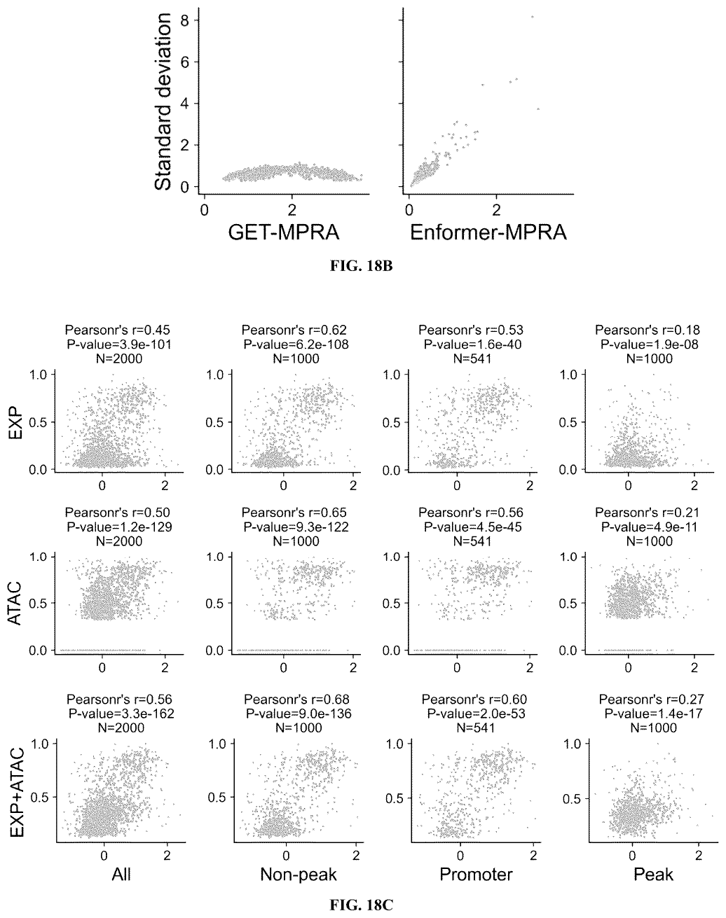

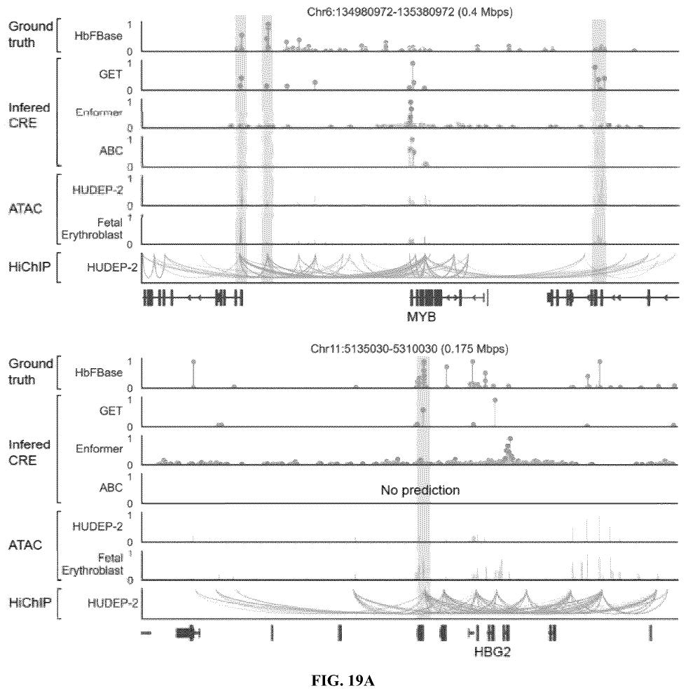

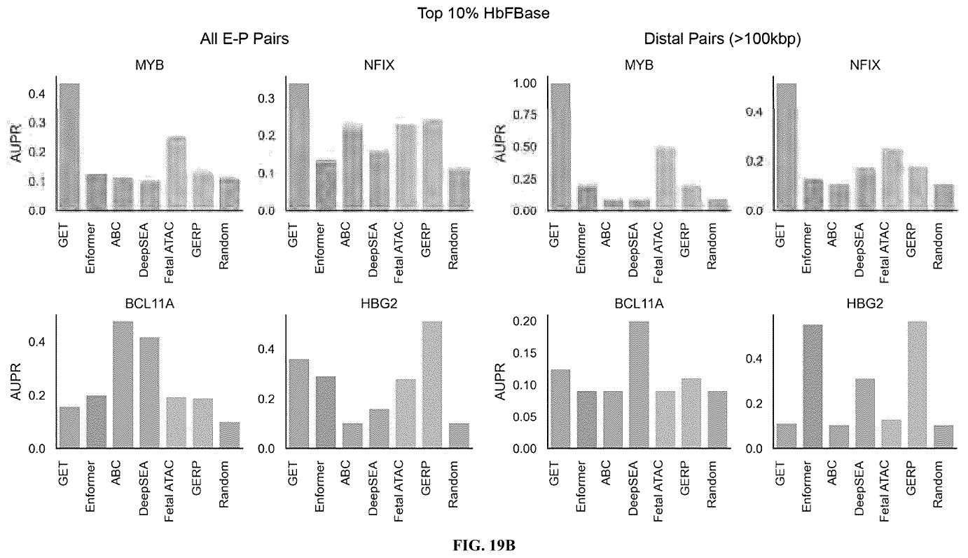

Given GET's demonstrated adaptability, its applicability to other experimental assays was explored. As a representative example, chromatin accessibility was chosen. First, a GET model was pretrained using only motif binding scores in accessible regions, and then fine-tuned it to predict quantitative K562 peak-level chromatin accessibility from both ENCODE OmniATAC (Listì, F. et al. Hemoglobin 42, 103-107 (2018)) or NEAT-seq (Zhou, J. & Troyanskaya, O. G. Nature Methods 12, 931-934 (2015)) data using a split chromosome evaluation method. Remarkably, the model achieved a Pearson correlation exceeding 0.98 for the left-out chromosome 11 (FIG. 18A). This might be attributed to the intrinsic association between chromatin accessibility and local DNA sequence patterns.

### Example 6

### Zeroshot GET Prediction of Expression-Driving Regulatory Elements in New Cell Type

Building on the versatility of GET across diverse platforms and measurements, its capacity for zero-shot prediction of expression-driving regulatory elements in unseen cell types was examined. Lentivirus-based massively parallel reporter assay (lentiMPRA) provides a robust mechanism to test the regulatory activity of numerous genetic sequences by integrating them into the genome, thereby circumventing the limitations inherent in episomal MPRAs and ensuring relevant biological readouts in hard-to-transfect cell lines. Recently utilized to assess over 200,000 sequences in the K562 cell line, this experimental assay has created a comprehensive benchmark dataset for evaluating whether the GET model can identify regulatory elements in a cell-type specific context (Nasser, J. et al. Nature 593, 238-243 (2021); FIG. 11C).

In an in silico procedure akin to the lentiMPRA experiment, the GET model finetuned on K562 chromatin accessibility and expression profile, which has not seen any lentiMPRA data was employed. The sequences were then constructed for insertion, including both the regulatory sequence and minipromoters, randomly inserting these sequences across the genome. Utilizing the GET model, the activity of the mini promoter within the corresponding chromatin context was inferred and averaged over all insertions to obtain a mean readout indicative of the regulatory activity (FIG. 11C, Methods). Best performance is achieved when the mean expression readout was combined with GET-predicted accessibility of the inserted element (FIG. 18B).

Upon examination of the readout distribution for different types of elements (FIG. 11D), the predictions were found to be consistent with experimental data. Promoter sequences exhibited the highest GET-MPRA readout, followed by chromatin accessibility peak sequences, with heterochromatin sequences registering the lowest readouts, and control sequences spanning a wide range of readout values (FIG. 11D).

When benchmarking the model against Enformer, the previous state-of-the-art model that utilized 486 different types of functional genomics data of K562, including transcription factor and histone modifications chromatin co-immunoprecipitation sequencing (ChIP-seq), cap analysis of gene expression (CAGE), and chromatin accessibility measurements, it was discovered that the model made more accurate predictions overall (Pearson's r=0.56 versus 0.44), although Enformer outperformed in peak regulatory activity predictions, which can be attributed to its nucleotide-level modeling architecture and extensive training data specifically targeting K562. Overall, Enformer's predictions tend to have larger across genome variance (FIG. 18C). Ablation GET also presented significant advantages in computational cost. In fact, for this comparison, 2,000 elements were sub-sampled to complete the calculation with Enformer in 3 days. While using the same amount of computing time GET can screen all 200,000 elements.

### Example 7

### GET Accurately Identifies Cis-Regulatory Elements and Upstream Regulators

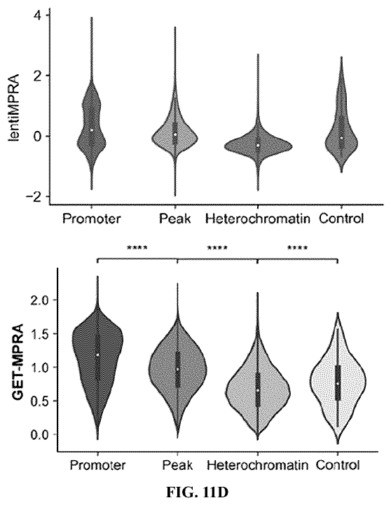

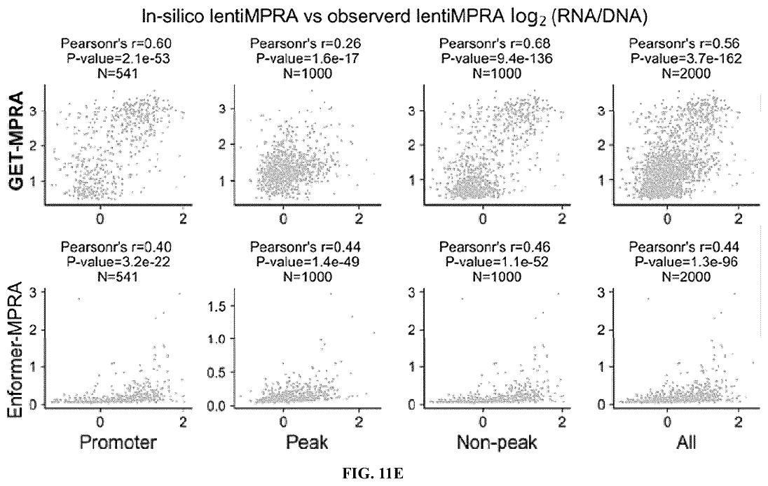

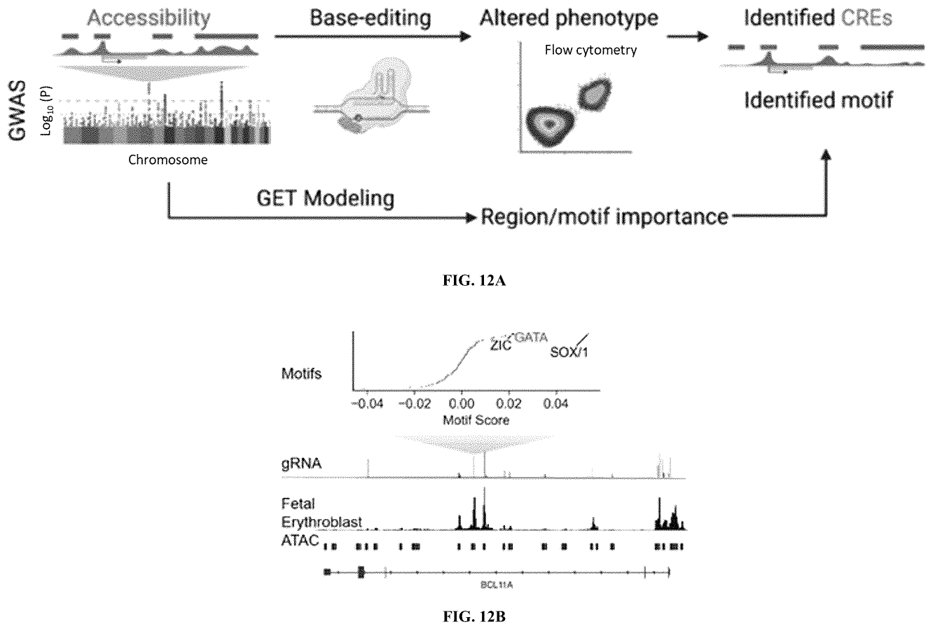

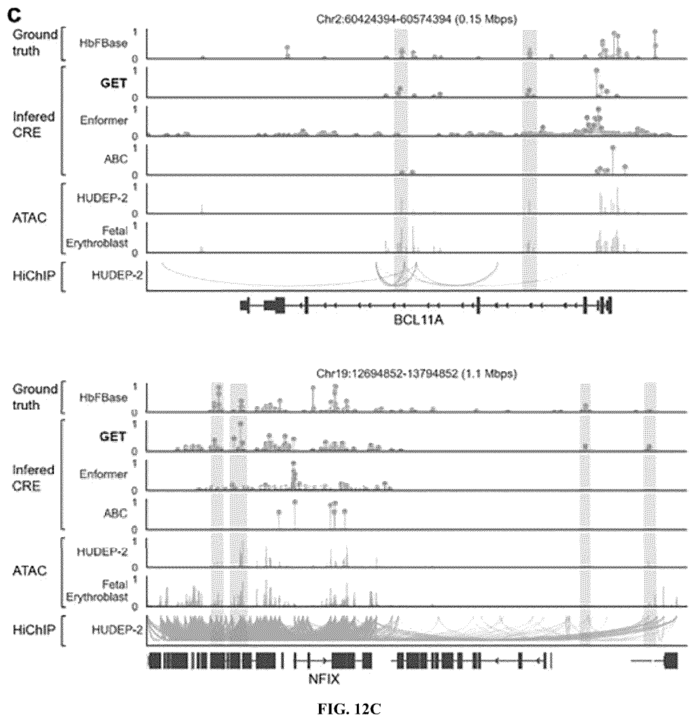

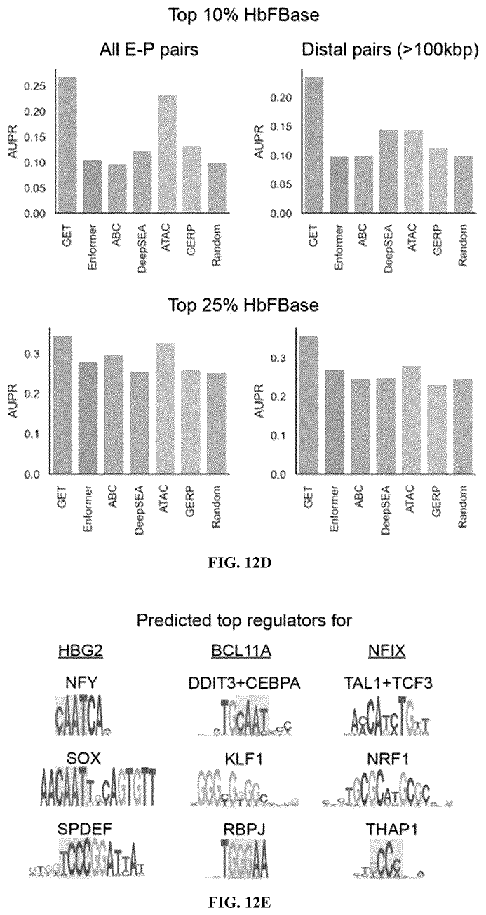

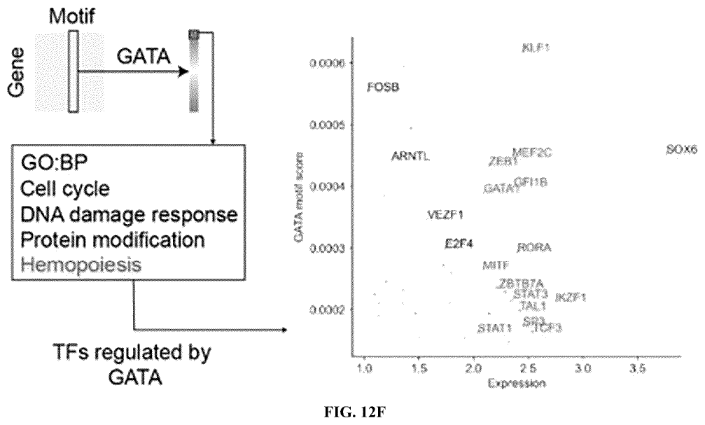

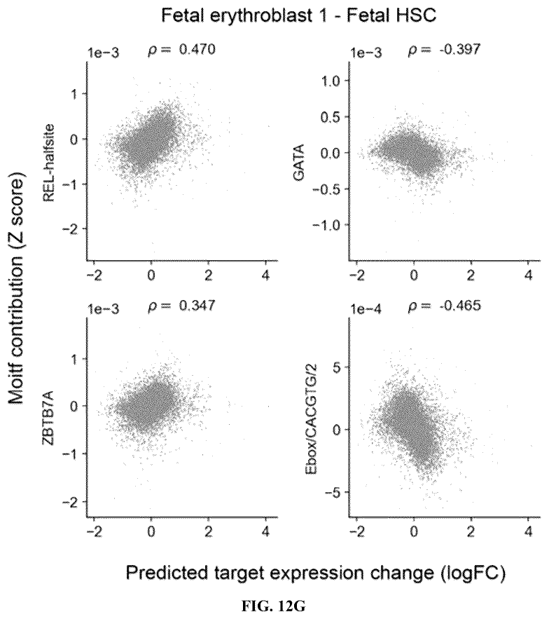

Single-cell multiome studies enable the identification of cis-regulatory elements (CRE) in specific cell types, offering potential phenotype intervention targets. Traditional peak-to-gene workflows largely depend on correlating multiome ATAC-seq and RNA-seq counts, with regulator identification necessitating additional filtering by transcription factor (TF) expression. Such methods are limited in comprehensiveness due to the nonlinear relationship between accessibility and transcription level and sparsity of single cell data, usually can only produce results for thousands of genes. Through model interpretation techniques (Methods), region/motif contribution scores for expressed genes across cell types can be efficiently derived, producing results for virtually all genes in even less abundant cell types (˜1,000 cells). Focusing on Fetal erythroblasts, published genome base-editing data was leveraged to investigate four known fetal hemoglobin regulating loci27 (BCL11A, NFIX, KLF1, HBG2, where first three are transcription factors genes and HBG2 encodes a fetal hemoglobin subunit, FIG. 12A).

Applying GET to fetal erythroblasts yielded interesting insights into the regulation of fetal hemoglobin. The central role of the GATA transcription factor was rediscovered, which, via its binding to an erythroid-specific enhancer, orchestrates the expression of BCL11A, a known modulator of hemoglobin regulation. Interestingly, GET also highlighted the role of the SOX family of transcription factors in this enhancer, which were previously linked to fetal hemoglobin but not known to function through this specific enhancer.

Examining all four loci, BCL11A, NFIX, KLF1, and HBG2, GET was benchmarked against established models like Enformer (Granja, J. M. et al. Nat Genet 53, 403-411 (2021)), DeepSEA (Baubec, T., et al., Cell 153, 480-492 (2013), and Activity-by-Contact (ABC; Domcke, S. et al. Nature 528, 575-579 (2015) and Shimizu, S., et al. Journal of Machine Learning Research 7, 2003-2030 (2006)). Distinctly, GET outperformed these counterparts, especially in detecting long-range enhancer-promoter interactions (FIGS. 12C-12D and 19A-19B). While enhancer chromatin accessibility is predictive of regulatory activity for proximal enhancer-promoter relationships, its precision diminishes for long-range interactions. Alternative evaluations using different functional enhancer thresholds (top 10% or 25% mean HbFBase, the gRNA enrichment score defined in the original study27) reaffirm GET's precision in this scenario (FIG. 12D).

GET is able to extract overall motif importance across cis-regulatory elements (CREs) for specific genes. For HBG2, BCL11A, and NFIX, the top motifs identified were consistent with their known transcriptional regulators or hematopoietic transcription factors (FIG. 12E). For instance, significance of NFY and SOX motifs for HBG2 and the reaffirmation of KLF1's influence on BCL11A was found. Additionally, for NFIX, GET adeptly pinpointed the involvement of TAL1, a known GATA1 binding partner and hematopoietic factor.

To determine downstream targets for specific regulators, an in silico analysis was developed, taking the GATA motif as a case study. Using the GET motif contribution matrix, the top 10% of genes influenced by the GATA motif were spotlighted. Notably, aligning with GATA1's status as a master regulator of erythroid development, the hematopoiesis biological process was enriched (Enrichment P-value=7.6×10⁻⁴ with multiple testing correction, FIG. 12F and Methods) within this gene set. Delving further, the identification of transcription factors within this set laid the foundation for an erythroblast-specific transcription factor regulatory framework centered on GATA. Recognized erythroid lineage transcription factors like KLF1, GATA1, TAL1, and IKZF1 were predicted to be regulated by the GATA motif, underscoring GATA's pivotal role in a multifaceted regulatory network, in line with existing literature.

To assess GET's capability to detect significant regulatory alterations across different cell states, the differential expression between Fetal erythroblast and fetal hematopoietic stem cells (HSC) was the focus. Genes that mark the lineage differentiation should receive more gradient from lineage specific transcription factors than those that are indifferent across lineages. The results confirmed this, as substantial Spearman correlation between the motif contributions in erythroblast and the differential expression log fold changes for several erythroblast-related transcription factors, including GATA, ZBTB7A, and MZF1 (FIG. 12G).

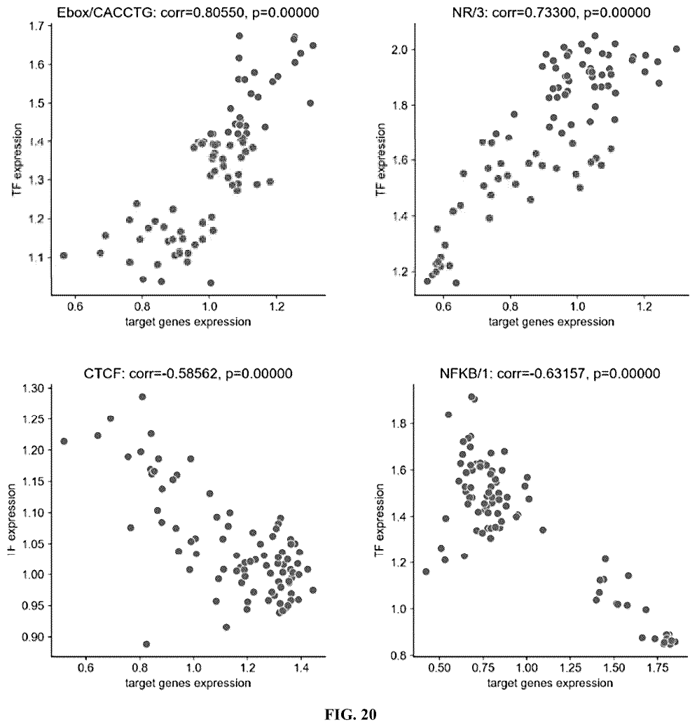

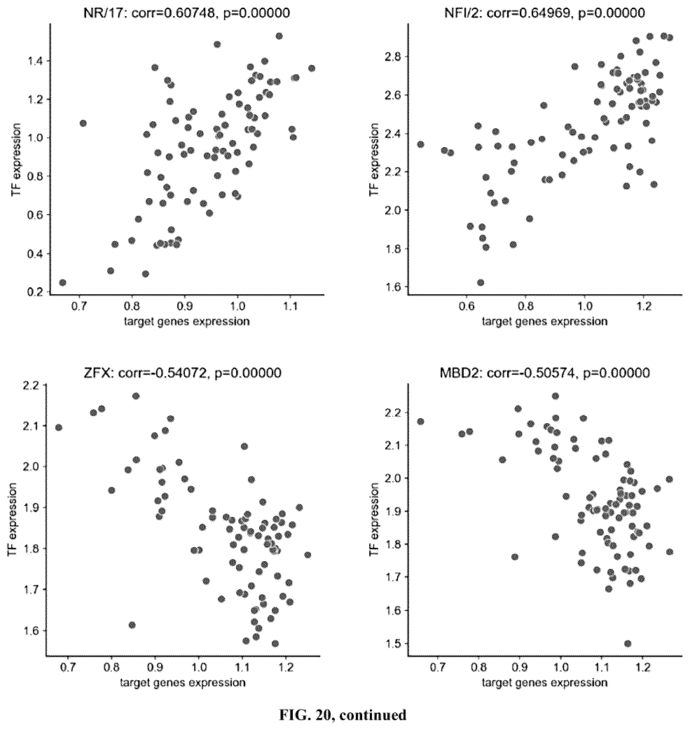

Extending the analysis to encompass all fetal and adult cell types, it was found that for certain well-known regulators, such as CTCF, MBD2, NFKB, and NFI, there exists a significant correlation between the mean expression of inferred target genes and the mean expression of all regulators within the corresponding family (e.g., NFIA, NFIB, NFIC, NFIX for the NFI motif, FIG. 20). Overall, GET possesses the ability to learn meaningful regulatory information that is naturally transferable between cell states.

### Example 8

Cell-Type Specific Regulatory Insights Through Cross-Cell-Type Embedding with Get

Utilizing a cross-cell-type architecture, GET is configured to extract the regulatory context for genes spanning various cell types, embedding them within a shared high-dimensional space (Methods). In order to further visualize what GET learns from different cell types, the embedding of different layers of GET was explored. Embedding from final layers correlates well with expression levels, while earlier layers are more indicative of differences in regulatory grammar.

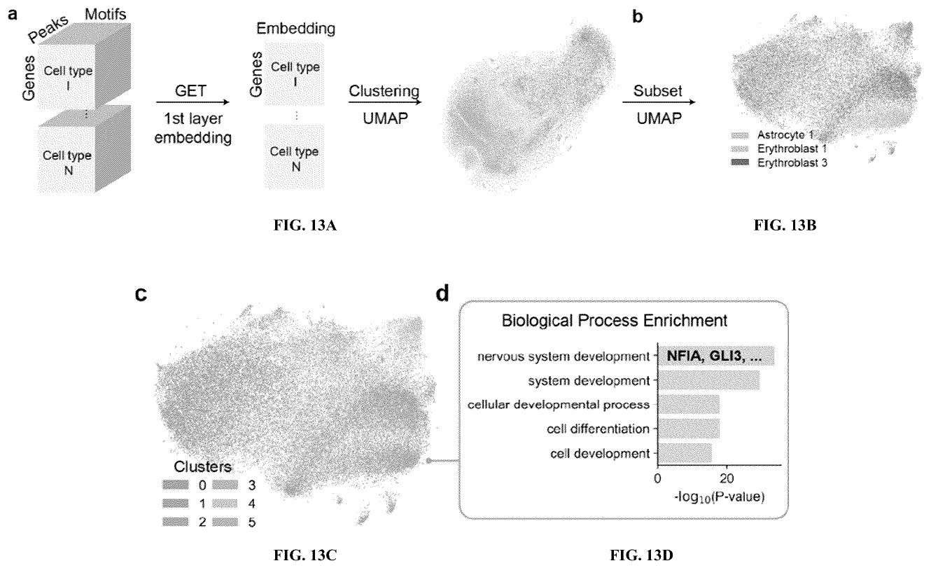

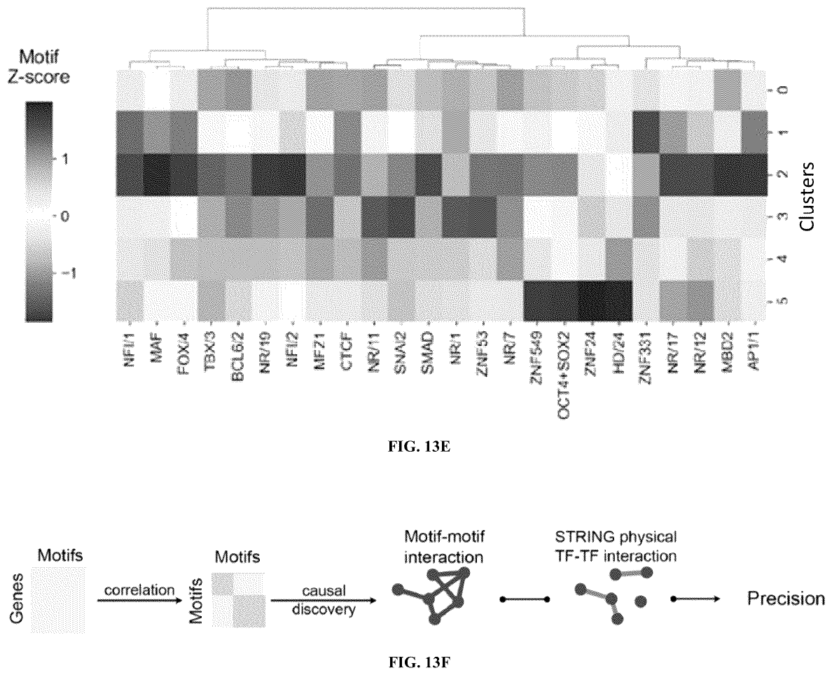

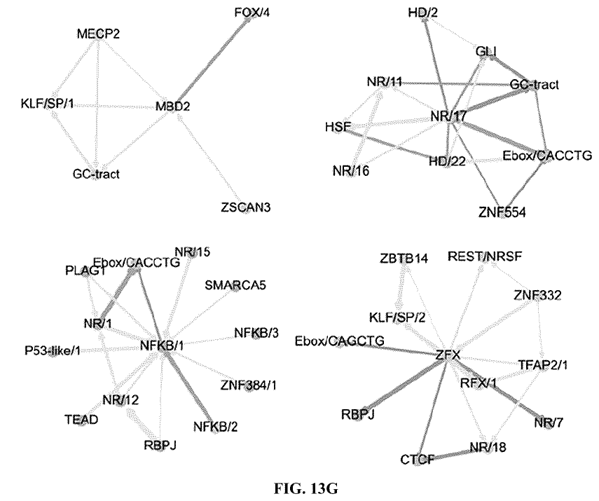

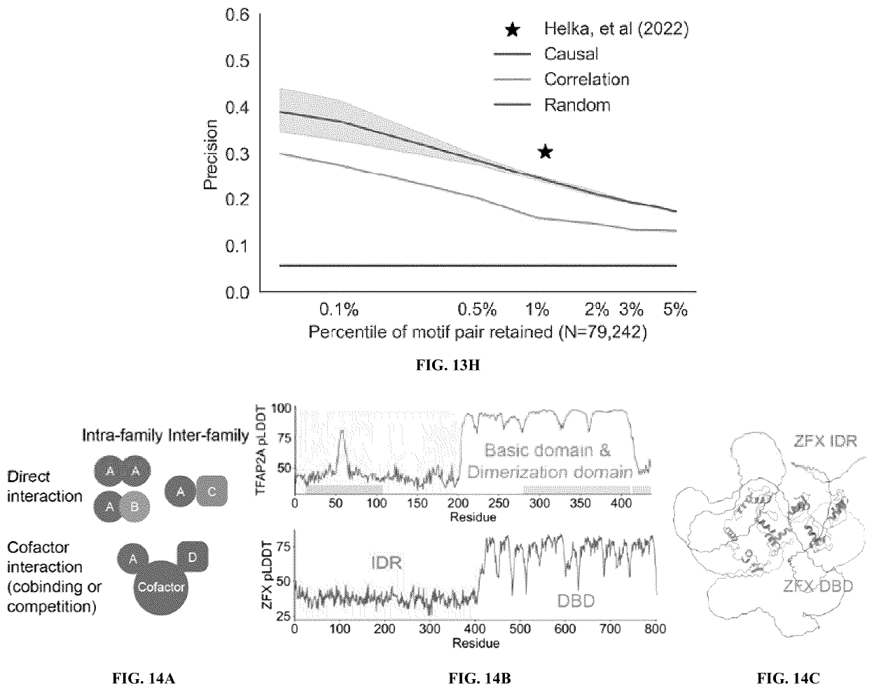

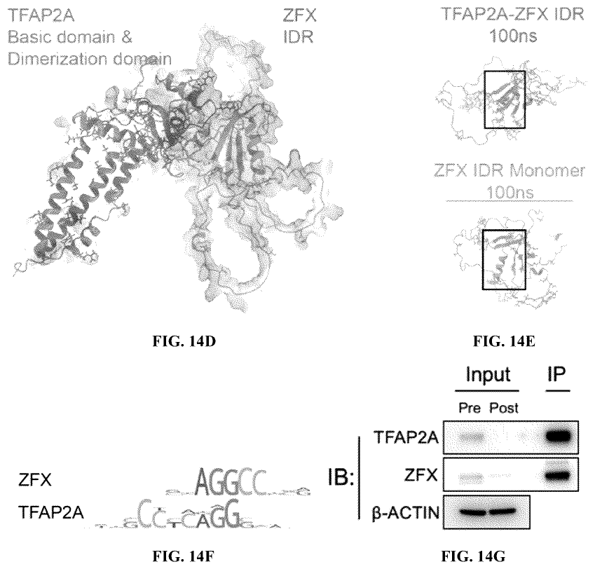

To investigate whether the embedding tied to regulatory grammar retains cell type-specific information, the first transformer layer's embedding were gathered for all promoters across cell types. This allows capture of not only the motif information within the promoter but also within the cis-regulatory elements (CREs) due to the attention mechanism employed. Intriguingly, a Uniform Manifold Approximation and Projection (UMAP) visualization of randomly sampled embeddings showed motif separation but no cell type differentiation, suggesting that, with a sufficient number of cell types, most regulatory grammar is shared across cell types, although they may be instantiated on different genes (FIG. 13A).

Nonetheless, when the embeddings were subset to only three specific cell types (fetal astrocyte and two fetal erythroblast subclusters), the UMAP exhibited distinct clusters for astrocytes and erythroblast genes (FIG. 13B). This result further corroborates that GET is proficient at discerning cell-type-specific regulatory information.

Delving further into the astrocyte-specific gene cluster (cluster #2 in FIG. 13C), this gene set is particularly enriched in the development of the nervous system and includes astrocyte transcription factors such as NFIA and GLI3 (FIG. 13D). Moreover, a comparison of motif contribution across clusters revealed a higher presence of NFI motifs in the astrocyte-specific cluster (FIG. 13E), shedding light on the unique regulatory program within astrocytes.

### Example 9

### GET-Based Causal Discovery Identifies Potential Transcription Factor-Transcription Factor Interaction

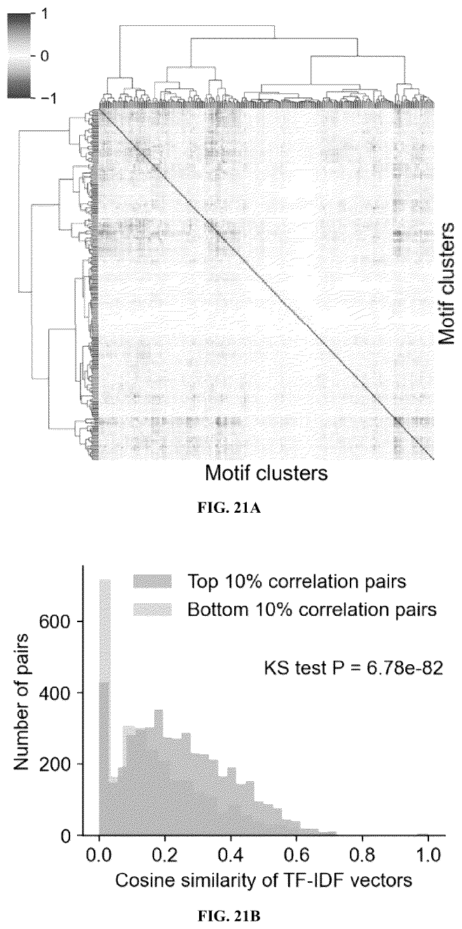

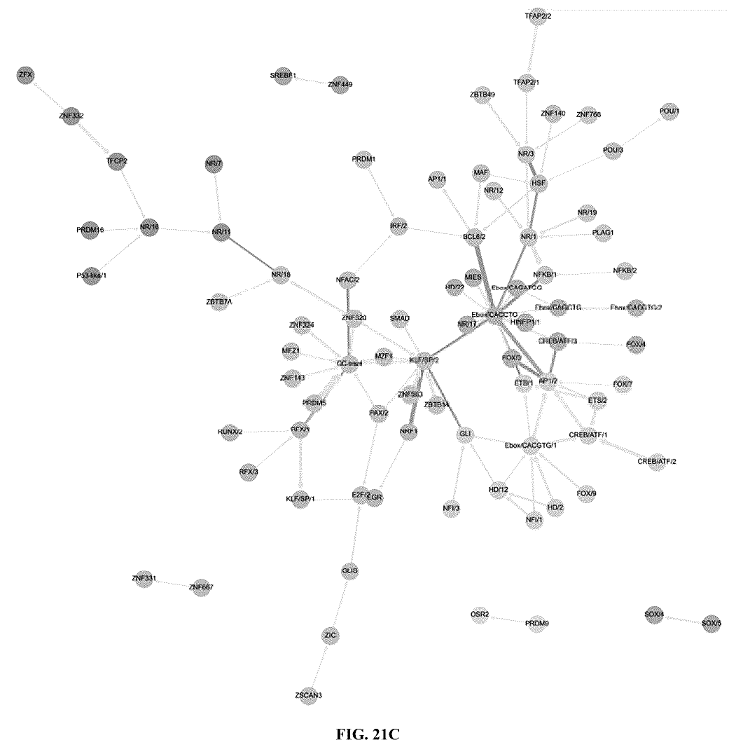

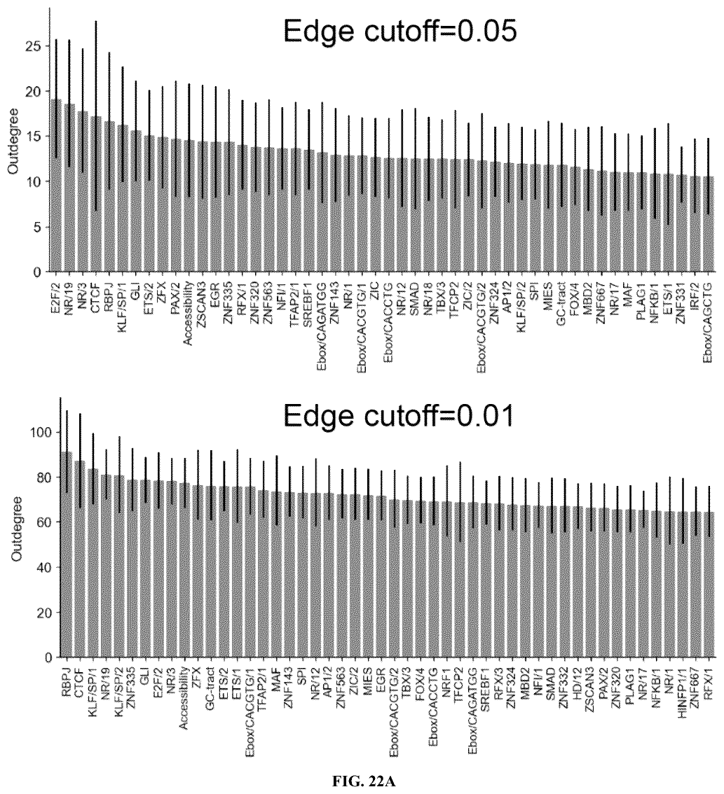

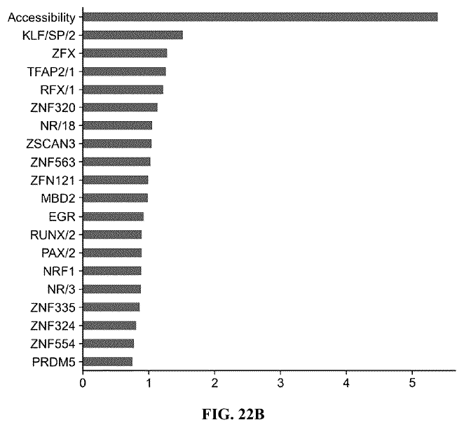

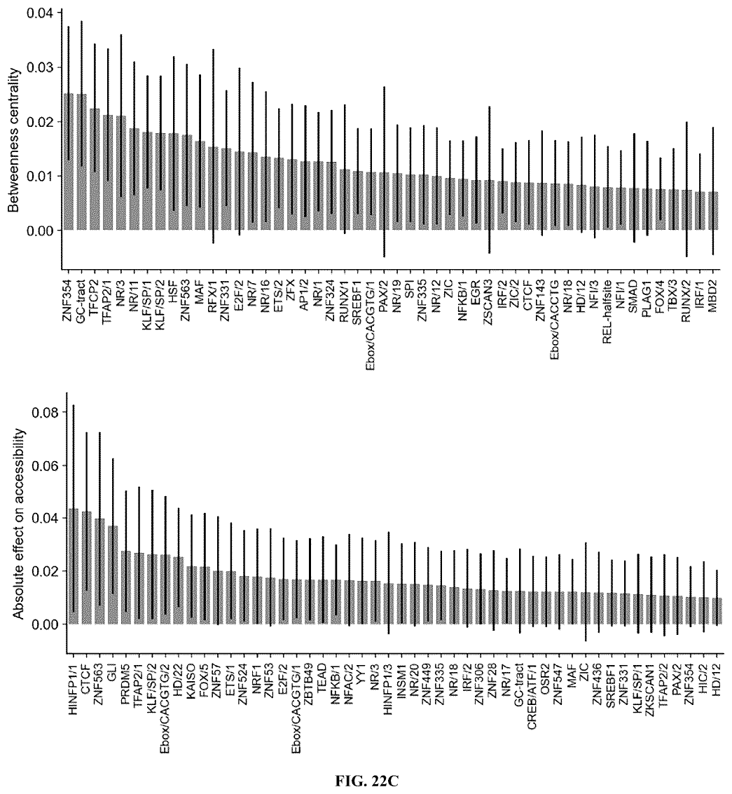

Given GET's proficiency in elucidating intricate regulatory mechanisms across diverse cellular contexts, next investigated was whether it learns transcription factor-transcription factor functional interactions implicitly. Using a cell-type agnostic gene-by-motif matrix (Methods), the correlation between different motif vectors was evaluated (FIG. 12A). High correlation may represent common genomic targets between different transcription factors. Remarkably, the transcription factor pairs with correlation values in the top decile are more likely to participate in the same biological functions compared to those in the bottom decile (FIG. 21B, Kolmogorov-Smirnov test, P-value 6.78×10⁻⁸²). For example, MBD2 and MECP2, a high correlation transcription factor pair, act both as readers of DNA methylation.

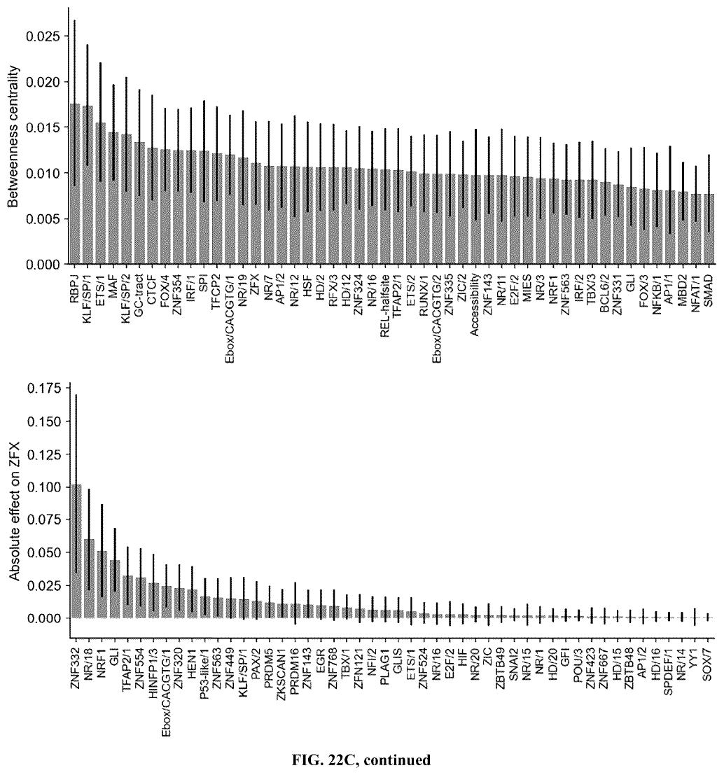

The investigation of the motif-motif interactions was further extended by utilizing a causal discovery algorithm, Linear Non-Gaussian Acyclic Model (LiNGAM), to derive a directed acyclic graph from the cell-type agnostic gene-by-motif matrix (Methods). The consequent network, displaying interactions with an absolute value greater than 0.1 for clarity in visualization, can be seen in FIG. 21C. Interesting, factors such as CTCF, KLF/SP/2 (GC rich motif), TFAP2/1, ZFX, RBPJ, Accessibility, and methylation-associated E2F were identified as having the largest outdegree in the causal network across diverse cell types (FIG. 22A, Methods), indicating the general importance of these factors in transcription regulation. The GET model trained using both quantitative ATAC signal and motif binding score as input got similar top out-degree transcription factors (FIG. 22B).

Four subnetworks are presented in FIG. 13G as examples. Notably, MBD2 and MECP2 has negative interaction with a promoter-enriched motif, GC-tract, which aligns with the well-known repressive effect of promoter methylation on gene expression. The other three networks centered around NR/17 (Representative transcription factor: ESR1), NFKB/1 (Representative transcription factor: RELA), and ZFX exemplify the diverse information GET has learned. For example, the pair NR/17-Ebox/CACCTG highlights a functional regulatory complex ESR1-ZEB1. NR/17-GLI is also supported by the known physical interaction between ESR1 and GLI3. NFKB/1-Ebox/CACCTG has a strong interaction with negative effect size, while their representative transcription factor, RELA and SNAI1, has been shown to be interacting using co-immunoprecipitation. ZFX is positively linked to TFAP2/1, and has been shown to co-localize with TFAP2A using ChIP-seq. NFKB/1 and NFKB/2 are dimer motifs of NFKB family transcription factors with NFKB/1 motif specifically from NFKB1, NFKB2 and NFKB/2 motifs also contributed by REL, RELA and RELB. The strong link between these two motifs are thus expected.

To quantitatively assess the overlap with currently known physical interactions between transcription factors, the GET motif-motif interaction network was compared with STRING v11 database (Methods). The results show a precision (true positive rate) of 5.6% by random chance. However, by selecting the top 1% (793 pairs) causal or correlation pairs from GET's predictions, precisions of 25.2% and 15.9%, respectively, were achieved, confirming the advantage of this approach. As a comparison, a recent mass spectrometry-based transcription factor-transcription factor interaction study (Gu, Z. et al. PAX5-driven Subtypes of B-progenitor Acute Lymphoblastic Leukemia. Nat Genet 51, 296-307 (2019)) reaches 30.4% precision with top 1.25% (990) pairs. This reflects the incompleteness of annotated transcription factor-transcription factor interactions and highlights the GET-predicted motif pairs as a valuable orthogonal information for this task.

### Example 10

### A Structural Catalog of Human Transcription Factor and Coactivators

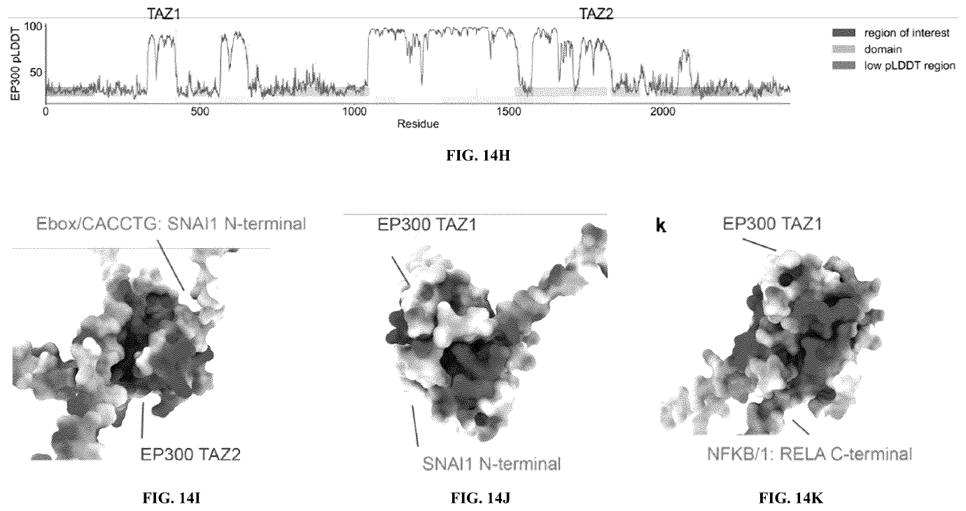

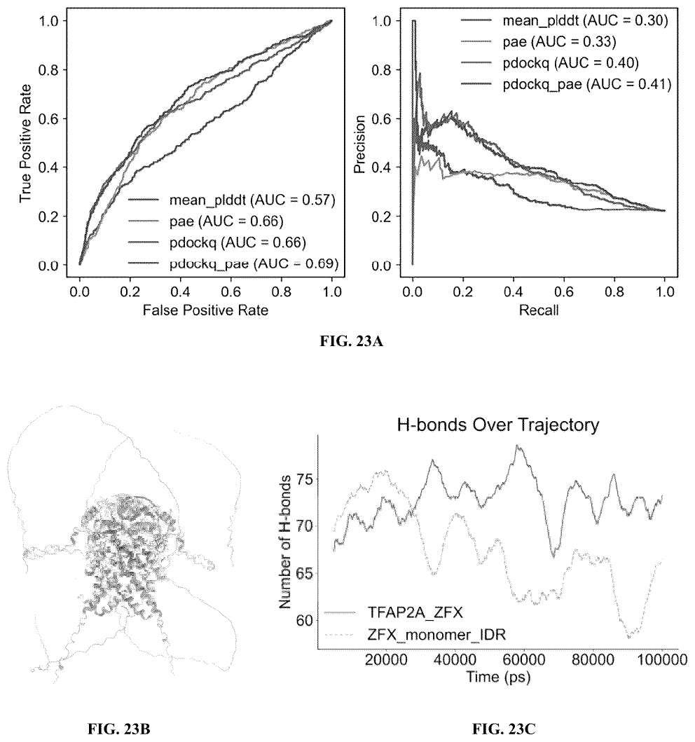

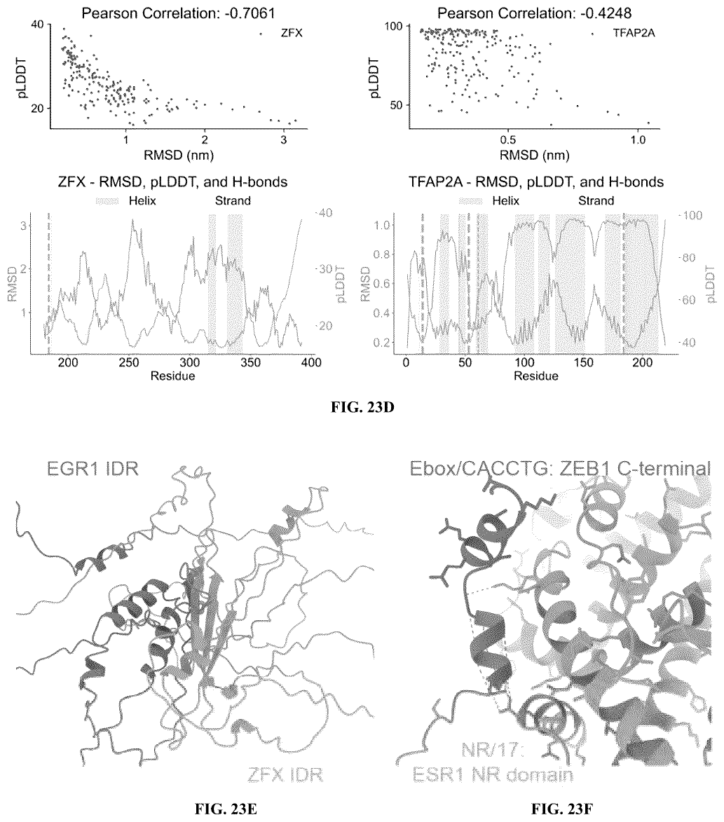

With the predicted causal motif interaction network predicted by GET, a structural catalog of the human transcription factor interactome was built using Alphafold2. It was started by categorizing transcription factor-transcription factor interactions into several different catalogs: Direct interaction, which includes homodimer, intra-family heterodimer, or inter-family heterodimer, and cofactor-mediated interaction, which may encompass both cooperative and competitive binding (FIG. 14A). Starting from the most straightforward intra-family interactions, all dimeric structure predictions of more than 1,700 known human transcription factors were first acquired. To evaluate whether Alphafold2 predictions reflect true interactions, the result on predicting whether a transcription factor family can act as an ‘intra-family binder’ was assessed based on the heuristic that intra-family binders should have a higher chance to form homodimers due to very similar structured domains. Alphafold can reach an area under the receiver operating characteristic (AUROC) of 0.69 and an average precision (AUPR) of 0.41 (FIG. 23A). The accuracy of AlphaFold dimer prediction is exemplified by the perfectly aligned TFAP2A structure to experimental results (FIG. 23B), even though there is no other similar template in PDB.

With AlphaFold2's established ability to predict unseen multimer structures, whether the disordered region in the structure could fold upon binding to partners was questioned. Based on causal discovery predicted transcription factor-transcription factor interactions, potential structural interactions were identified using AlphaFold2. Taking TFAP2A and ZFX as an example, both proteins were segmented into four distinct structured or disordered domains based on predicted local distance difference test (pLDDT) (FIG. 14B) and the multimer structure of all pairwise combinations between these segments was predicted. Remarkably, the originally unstructured ZFX intrinsically disordered region (IDR) (FIG. 14C) folded into a well-defined multimeric structure when paired with TFAP2A structured domains, mainly driven by electrostatic interactions (FIG. 14D).

To provide another line of evidence, molecular dynamics simulations were employed (Methods), discovering that the monomer IDR exhibited a more collapsed structure after 100 ns (FIG. 14E) and fewer inter-chain hydrogen bonds (FIG. 23C). Moreover, the per-residue pLDDT of ZFX IDR and TFAP2A in the multimer structure correlated strongly with residue instability, as measured by root mean squared distance (RMSD; FIG. 23D), aligning with previous studies indicating AlphaFold's implicit learning of protein folding energy functions. To validate the predicted interactions between these two proteins, co-immunoprecipitation experiments were performed. As shown in FIG. 14F, these experiments were able to pull down ZFX using a TFAP2A antibody.

When extending the method to negative effect pairs such as SNAI1 (Ebox/CACCTG) and RELA (NFKB/1), the absence of robust structural interactions, despite previous physical evidence, led to exploration of cofactor-mediated interactions (FIG. 14I). Both transcription factors are known to physically interact with EP300, and the predicted structures underscored electrostatic interactions with EP300's TAZ1 and TAZ2 domains (FIG. 14J). This concurs with existing studies on the electrostatic binding of transcription factor IDR to EP300 TAZ domains.

Broadening the study, the procedure was applied to the top 5% transcription factor pairs in each cell type (totaling 1,718 transcription factor pairs or 24,737 pairs of transcription factor segments, see Methods) as predicted through GET-based causal discovery and built a structural catalog of transcription factor interactions. Interestingly, the folded conformation of ZFX IDR can also be seen in other transcription factor pairs, for example EGR1 IDR-ZFX IDR (FIG. 23E). The previously mentioned interaction between ESR1 and ZEB1 could be driven by a confident structural interaction between the ZEB1 C-terminal IDR and ESR1 NR domain (FIG. 23F).

### Example 11

### GET Uncovers Mechanism of Germline Mutation in Disorder Region of Transcription Factors

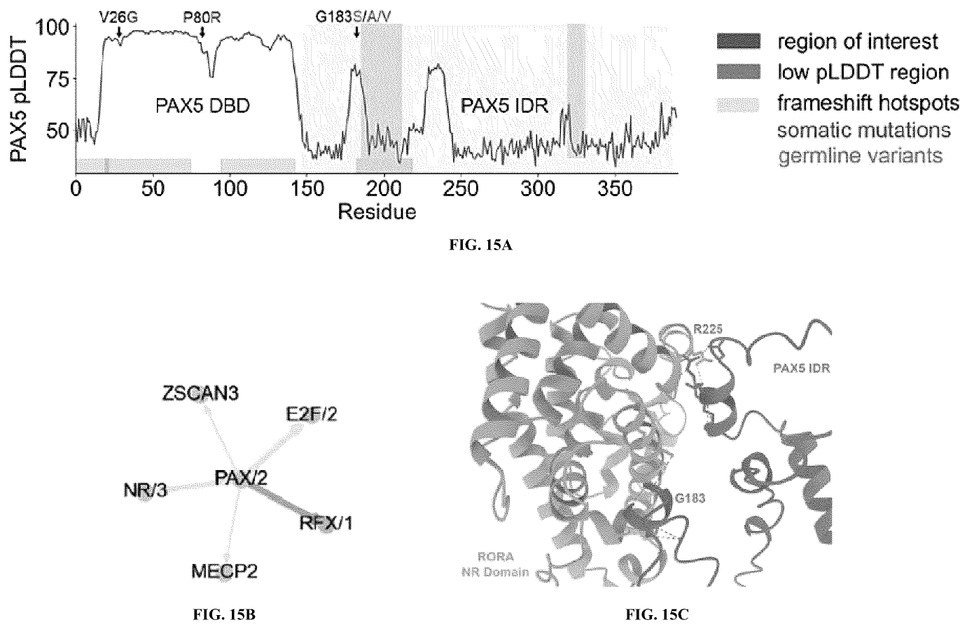

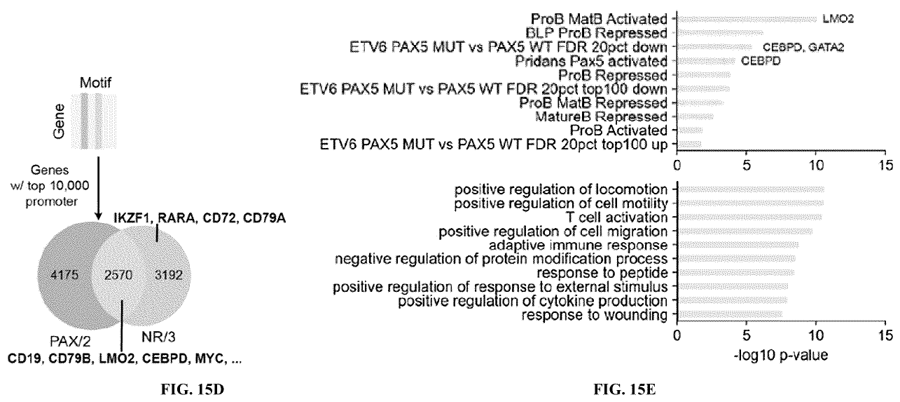

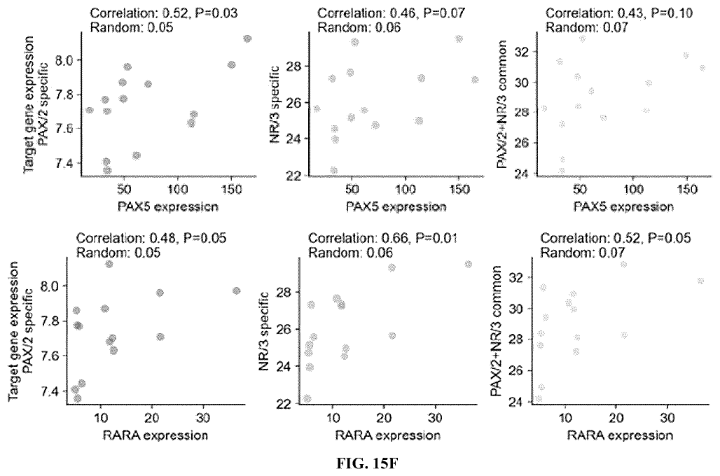

To demonstrate the utility of information provided by the GET Catalog, a case study was performed on PAX5, a driver transcription factor of B-cell precursor acute lymphoblastic leukemia (B-ALL). B-ALL is the most frequent pediatric malignancy, and somatic genetic alterations (deletions, translocations and mutations) in PAX5 occur in approximately 30% of sporadic cases. While most PAX5 somatic missense mutations affect the DNA-binding domain (V26G or P80R), G183S is a recurrent familial germline mutation that confers an elevated risk of developing B-ALL. Somatic mutation of G183 and frameshift in a nearby hotspot is also seen in B-ALL patients. Although the pLDDT plot of PAX5 highlights G183 and the octapeptide domain as a small peak in the entire intrinsically disordered region, its functional role remains elusive (FIG. 15A). To probe this, potential interaction pairs involving PAX5 (PAX/2 motif) in fetal B lymphocytes (CXCR5+) were first explored. Promising interactions were identified with several transcription factors including E2F3, MZF1, MECP2, NR4A2, RFX3, and RORA (NR/3 motif, FIG. 15B). Subsequent exhaustive segment interaction screening revealed a novel interaction between the RORA nuclear receptor (NR) domain and the octapeptide domain of PAX5 (FIG. 15C). The G183 residue is close to the binding site alpha helix where a mutation to Serine or Valine might introduce spatial clash. This interaction was further corroborated by positive affinity purification-mass spectrometry (AP-MS) data of their paralog PAX2-NR2C2, as both the PAX5 octapeptide domain and NR domain are highly conserved and structurally similar across their paralogs.

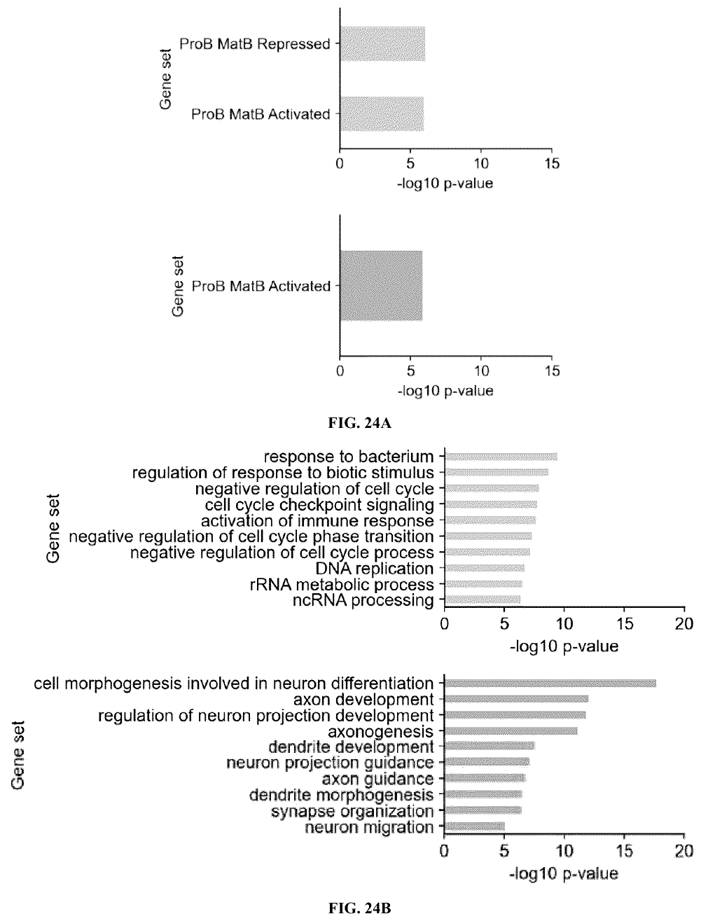

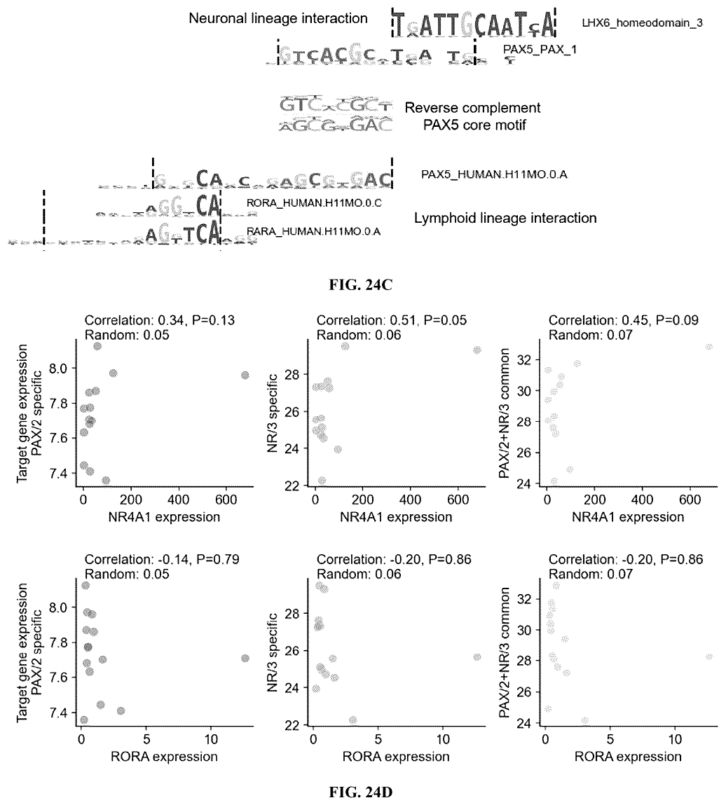

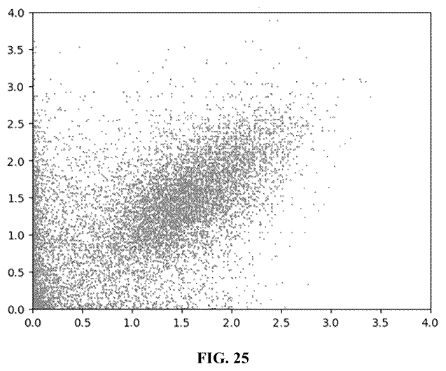

To elucidate whether PAX/2 and NR/3 motif co-regulate genes, the top 10,000 promoters predicted to be most influenced by them were examined. The analysis uncovered a set of 2,570 genes commonly regulated by both, including surface markers like CD19 and CD79B, as well as known oncogenes implicated in B cell acute lymphoblastic leukemia (B-ALL) including MYC, CEBPD, LMO2, although these oncogenes are also predicted to be strongly repressed by IKZF1 (IKAROS tumor suppressor, with ZNF143 motif), and are not highly expressed (FIG. 15D). Enrichment analysis revealed an overrepresentation of genes involved in leukocyte activation and genes affected by PAX5 perturbation during B cell differentiation, aligning with previous work on the G183S mutation (FIGS. 15E and 24A-24B). On the other hand, the genes that are specifically regulated by PAX/2 or NR/3 are enriched in neuronal pathways and cell cycle respectively. These results are corroborated by the sequence pattern of PAX/2 motif which contains a partial RARA/RORA motif, while another PAX5 motif, PAX/1, contains a partial LHX6 motif which is a neuronal lineage transcription factor (FIG. 24C).

Finally, patient tumor RNA-sequencing data was used to validate the findings. Using data from B-ALL patients without PAX5 somatic coding mutations, significant correlations (P<0.05) was found between both PAX5 and RARA/NR4A1 (paralogs of RORA with NR/3 motif) expression levels and the expression of predicted target genes (FIGS. 15F and 24D), further supporting the role of the PAX5-nuclear receptor interaction in lymphoma transcriptional programs. In sum, the analysis suggests that the PAX5 G183S germline mutation may confer B-ALL-specific risk by disrupting interactions between the PAX5 intrinsically disordered region and the nuclear receptor domains of other transcription factors, thereby leads to oncogenic transcriptional programs.

### Example 12

### Zero-Shot Application of Astrocyte-Leave-Out Fetal-Data-Trained Model

Infrastructure was built to on-the-fly preprocess 10× genomics data (single cell ATAC and RNA-seq data) into suitable format for model input while ensuring comparability between different datasets and keeping nucleotide-level information.

### Methods

### ATAC-Seq Data Processing

Pseudobulk To get the chromatin accessibility score for each region, the scATAC-seq count table and cell annotation table from each study were used. To group single cells into pseudobulk “cell types”, the louvain clustering result from each study was used. The cell type annotation of each cluster is used to define the biological cell type. A cell count >600 was empirically used as a threshold to select cell clusters with enough sequencing depth. A table of pseudobulk cell types used in the training process is compiled below.

Cell-type-specific accessible region identification For the identification of cell-type-specific accessible regions, the peak calling results from the original studies of each dataset were followed to obtain a union set of peaks. Subsequently, to compile a list of accessible regions specific to each cell type, peaks with no counts were filtered out.

In the context of the human fetal and adult chromatin accessibility atlas, the peak set produced by Kai Zhang et al. (Cell 184, 5985-6001.e19, (2021)) was employed, incorporating the fetal chromatin accessibility atlas originally published by Silvia Domcke et al. (Science 370, (2020)). A version of fetal-only GET model was also trained using the original peak calling and cell type annotation from Silvia Domcke et al. (Science 370, (2020)), resulting in comparable expression prediction and regulatory analysis performance. For the 10× multiome data, the provided peak fragment count matrix was used. For the K562 NEAT-seq and bulk chromatin accessibility data, a more permissive version of peaks was called using MACS2, and different logTPM cutoffs were applied to the resulting peak set to select accessible regions. This accessibility-based data augmentation enhances the diversity of input data and fine-tunes the GET model for data from a single cell type. The code for processing is publicly available in the Github repository at atac ma data processing.

Accessibility Features In the study, the chromatin accessibility score for a specific genomic region is defined by the count of fragments located within that region for a given cell type pseudobulk. To enhance the model's generalizability, these counts are further normalized through the logTPM (Log Transcripts Per Million) procedure. Specifically, let t be the total fragment count in a pseudobulk, and cᵢ be the fragment count in region i. Then, the accessibility score sᵢ is computed as:

$${s}_{i} = {log}_{10} ( \frac{{c}_{i}}{t} + 1 ) , t = {\sum }_{i} {c}_{i}$$

For the majority of the regulatory analysis, the ‘Without-ATAC’ version of the GET (Gene Expression Tracking) model is utilized to comprehensively evaluate the regulatory influences exerted by transcription factors. In both the training and inference phases of this specific model version, the accessibility scores are uniformly set to 1 if the region is identified as a chromatin accessibility peak. This equates to assuming binary chromatin accessibility states within the studied scenario.

Motif features To calculate the motif binding score within a specific genomic region, the corresponding sequence is scanned against the hg38 reference genome. This procedure involves utilizing 2,179 transcription factor motif position-weighted matrices (PWMs), as previously compiled by Vierstra et al. (Nature 583, 729-736, (2020)), accessible at Vierstra's resources. For the scanning process, the MOODS tool is used with default threshold.

More specifically, to represent sequence information while mitigating feature redundancy, a specialized motif scoring process is implemented. Building on the prior research of Jeff et al., these 2,179 motifs are categorized into 282 motif clusters, a classification determined by PWM similarity. By using this established clustering definition, nucleotide-level motif matches that are redundant are eliminated, retaining only the match with the highest score within overlapping matches belonging to the same motif cluster.

Subsequently, the scores of all non-overlapping motif matches within each motif cluster are summed, yielding one cumulative score for each of the 282 clusters. As a final step, motif binding scores for all regions within a given cell type are determined and subjected to min-max normalization across regions. This normalization facilitates model generalization and the training process, ensuring that each motif cluster's score is processed in a standardized manner.

Input data GET is designed to capture the interaction between different regions and regulators. To facilitate that, each input sample contains a certain number of consecutive accessible regions, mimicking the “reception field” of an RNA Polymerase II. Through previous experiment it was found that ideally the equivalent genome coverage should be around or more than 2 Mbp, a range where most of the chromatin contact happens. As a result, based on the current data prepossessing pipeline it was chosen to use 200 as the input region numbers for one training sample. Non-overlapping samples were acquired from the genome to use as the pretrain input, and from only the training chromosomes to use as the finetune input.

GET model is engineered to encapsulate the interactions between neighboring regions. To achieve this, each input sample encompasses a specific number of consecutive accessible regions, simulating the “reception field” of an RNA Polymerase II. Through empirical research, it was determined that an optimal equivalent genome coverage for this purpose is approximately 2 Mbp or greater, a span within which the majority of chromatin contacts occur. Consequently, in line with existing data preprocessing pipeline, 200 was selected as the quantity of input regions for a single training sample. Non-overlapping samples were extracted from the genome for pretraining, and exclusively from training chromosomes for the finetuning phase.

A GET model was also developed that can take nucleotide-level sequence and ATAC input, and jointly consider transcription factor expression and genomic/epigenomic data. The framework was also able perform inference mutation data to predict their impact on gene expression.

### RNA-Seq Data Processing

Cell Type Matching For experiments encompassing multiomics, the correspondence between accessibility and expression is inherently determined through cell barcodes. In pseudobulk cases, where accessibility and expression are assessed independently, cell type annotations are utilized to facilitate the mapping. Specifically, the fetal expression atlas from Cao et al. (Science 370, 10.1126/science.aba7721 (2020)) is employed for fetal cell types, while adult data is extracted from Tabula Sapiens (Science 10.1126/science.abl4896 (2022)). When several ATAC pseudobulk share the same cell type annotation, identical expression labels are assigned. This approach, while a compromise, is due to the current dearth of multiome sequencing data, a situation expected to change dramatically in the near future.

### Expression Values

Expression values are allocated to each region within the input. Constrained by poly-A scRNA-seq, only aggregated mRNA levels can be captured, resulting in values that are not reflective of the nascent transcription rate more closely tied to regulatory events. Nonetheless, these values furnish valuable cell-type-specific information. The process begins by intersecting the input region list with Gencode V40 transcripts annotation to pinpoint promoters, followed by the assignment of log count per million (CPM) values to regions corresponding to these promoters. All remaining regions are assigned a value of 0. Although this does not perfectly represent all transcription events happening in a cell, the zero label on non-promoter region helps in delivering informative negative labels to the model.

### Input Target

In alignment with the 200×283 input matrix, the target input is a 200×2 matrix, symbolizing the transcription levels of the corresponding 200 regions across both positive and negative strands.

### Model Architecture

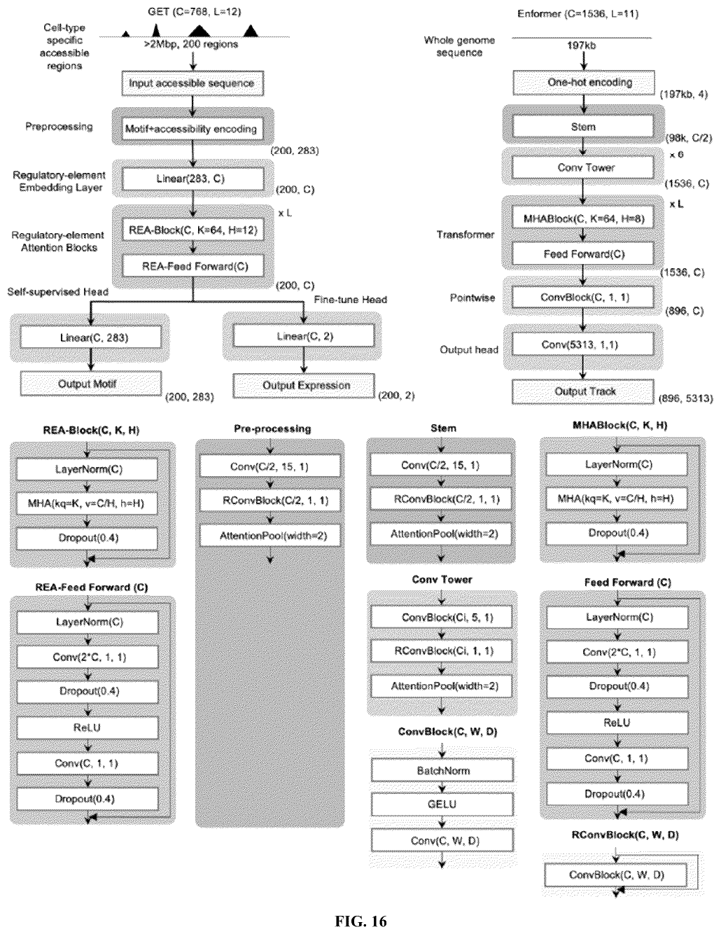

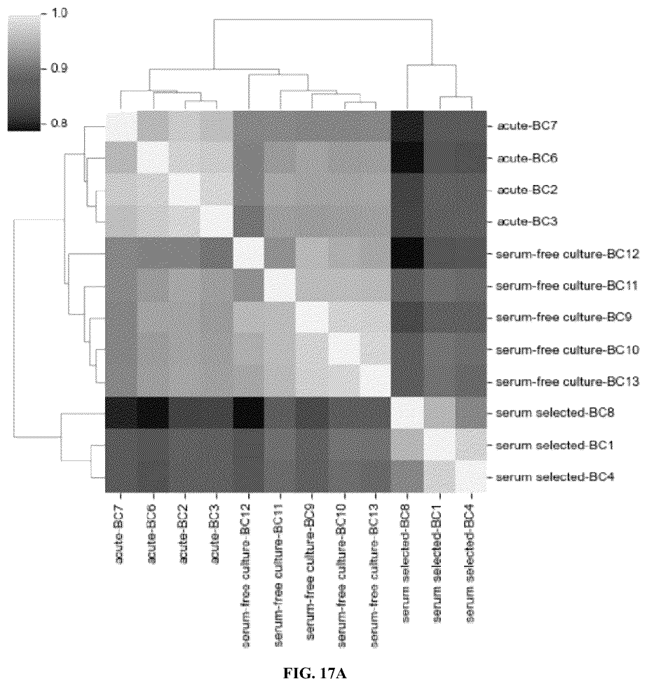

The GET architecture consists of three parts: 1) A regulatory element (RE) embedding layer, 2) 12 RE-wise attention layers, and 3) a linear layer as the expression prediction head (FIG. 16).

GET takes 200 regulatory elements, each with 282 motif binding scores and optionally one accessibility score as a sample as the input. As a result, the input is a 200×283 matrix. When it was chosen to not use the quantitative accessibility score, the 283-th column was set to 1.

Then the sample was fed into the RE embedding layer to generate the regulatory element embedding with a dimension of 768 for each peak. Since it was not wanted to lose information in the input of the original regulatory element, a linear layer was applied to capture the general information in the different classes of transcription factor binding sites. To learn the cis- and trans-interactions between regulatory elements and transcription factors, 12 RE-wise Attention (REA) layers with a multi-head attention mechanism was applied on the RE embeddings along the regulatory element.

Suppose Nₕ,dᵥ,dₖ denote the number of heads, the depth of values, and the depth of keys. The output from each head h is computed as

$${O}_{h} = Softmax ( \frac{{X}^{′} {{W}_{q} ( {X}^{′} {W}_{k} )}^{T}}{\sqrt{{d}_{k}}} ) ( {X}^{′} {W}_{v} )$$

where W_{q}, Wₖ ∈ [symbol] ^{(n×D)×d}ₖ, Wᵥ ∈ [symbol] ^{(n×D)×d}ᵥ are learnable linear transformations.

Then the output from each head h for the RE-wise Attention block was concatenated.

The Layer Normalization (LN), Feed-forward Network (FFN), and Residual Connections are finally utilized to generate the output for each layer. Thus, the mechanism behind the RE-wise attention block is summarized as:

$${z}_{l}^{′} = MHA ( L N ( {z}_{l - 1} ) ) + {z}_{l - 1} ; {z}_{l} = F F N ( L N {z}_{l}^{′} ) ) + {z}_{l}^{′}$$

where

$${z}_{l}^{′} , {z}_{l - 1}$$

denote the intermediate representation in the block l and the output from the block l-1. Two linear layers were applied with a GELU activation layer in the FFN layer.

The GET architecture is similar to the state-of-the-art model Enformer. However, the following changes helped improve and exceed its performance: GET uses the regulatory element (RE) embedding layer to capture the general information of regulatory elements in the different classes of transcription factor binding sites. Moreover, a masked regulatory element mechanism was utilized to learn the general cis- and trans-interactions between regulatory elements and transcription factors from different kinds of human cell types.

Specifically, a random set of positions was uniformly selected to mask out

$$M = {\{ {m}_{i} \}}_{i = 1}^{k}$$

with a mask ratio of r=k/n. The regions in the selected positions were replaced with a [MASK] regulatory element, and the masked input regulatory element is denoted as X^{masked}=(X, M, [MASK]) where

$$X = {\{ {x}_{i} \}}_{i = 1}^{n}$$

is the input sample with n regulatory elements. The training goal is to predict the original values of the masked elements M. Specifically, masked regulatory element embeddings X^{masked} are taken as input to GET, while a simple linear layer is appended as the prediction head. Therefore, the overall objective of self-supervised training is formulated as:

$$ℒ = 𝔼 ( {\sum }_{i ∈ M} - log p ( {x}_{i} \vert {X}^{masked} ) )$$

where xᵢ denote the masked region to be predicted.

### Training Scheme

Pre-training id conducted in the large-scale single-cell Chromatin accessibility data. Then the pre-trained model is fine-tuned on the Paired chromatin accessibility-gene expression data with the same Poisson negative log-likelihood loss function as Enformer. Expression values are represented as normalized transcript per million (TPM). The cell types between RNA and ATAC datasets are then matched by annotating cell type names and ignoring those that cannot be matched. To improve training stability, the expression values are log-transformed as log₁₀(TPM+1). The gene expression is mapped to accessible regions using the following approach: if a region overlaps with a gene's transcription start site (TSS), the gene's expression value is assigned to that region as a label; if a region overlaps with multiple gene's TSS, the expression values of the corresponding gene are summed up and used as the label of that region; if a region does not overlap with any TSS, the corresponding expression label is set as 0. Finally, each regulatory element is assigned to an expression target value.

The GET implementation is based on PyTorch (Paszke, A. et al. In Proceedings of Advances in Neural Information Processing Systems (2019)) framework. For the first training stage, AdamW (Loshchilov, I. & Hutter, F. In International Conference on Learning Representations(2019)) is applied as the optimizer with a weight decay of 0.05 and a batch size of 256. The model was trained for 800 epochs with 40 warmup epochs for linear learning rate scaling. The maximum learning rate was set to 1.5e-4. For the second fine-tuning stage, AdamW was used as the optimizer with a weight decay of 0.05 and a batch size of 256. The model was trained for 100 epochs.

### Model Evaluation

Pearson correlation, spearman correlation, and R2 are used to evaluate the prediction performance in all settings. For evaluation of cell type specific gene prediction, the observed and predicted log fold change are compared between two cell types using the same metrics.

Cross-cell-type expression prediction In cross-cell-type prediction setting, it was pretrained on ATAC-seq data from all cell types, and finetuned with expression data from only training cell types, hiding the evaluation cell type expression label from the model. On average, GET achieves 0.799 Pearson's r and 0.845 Spearman correlation across different leave-out-cell type settings. Furthermore, GET is able to get similar performance when chromosomes (chr4,chr14) are also leaved out (cell-type & chromosome leave-out, Spearman rho: 0.938, R2: 0.868, Pearson's r: 0.935).

Platform transfer prediction In order to transfer to a new sequencing platform, there are multitude of domain shift that need to be addressed. This including but not limit to: 1. Sequencing depth: as lower depth will lead to less captured peaks. It will also affect the signal to noise ratio in the accessibility quantification; 2. Peak calling threshold and software; 3. Technical bias due to different library constructing and sequencing method; and 4. biological differences

Due to these biases, there are hurdles to directly apply a model trained on one dataset to a new platform without finetuning. Thus, for a new dataset with multiple cell type available, a leave-out cell type approach of finetuning was taken. For a dataset of sorted cell types where only one cell type is available, leave-out chromosome training was used.

LentiMPRA zeroshot prediction The experimental procedure involves designing a library of lentivirus vector which contains both desired sequence elements and a minipromoter; then the vector will be randomly inserted on the genome through viral infection; the regulatory activity is then measure through sequencing and counting the log copy number of transcribed RNAs and integrated DNA copies. To simulate this approach using GET, the sequence element library was first collected and the vector sequence with mini promoter was constructed. Then followed the same data preprocessing procedure to get the motif score of the inserted elements. For each element, ‘in silico insertion’ is performed by sum up its motif score with an existing region on the genome. The +/−100 regions centered around the insertion region where then used as a input sample for GET to make expression prediction. The mean predicted expression (log₁₀TPM were multiplied with the predicted accessibility (using a GET model finetuned to predict ATAC logTPMs) as the predicted regulatory activity. For each region, 600 insertions were perform across the genome to match with the experimental insertion count. The GET model finetuned on K562 multiome and bulk ATAC- and RNA-sequencing data were used to perform the inference. For Enformer, the same analysis was performed, with the only difference in that the vector sequence is integrated to a random position on the genome and a 196,608 bp sequence centered is collected around the insertion site. Enformer is trained on 5,313 human epigenome track, with 486 experiments specifically for K562. To compute the regulatory activity, the output from the K562 CAGE track is selected, which is a quantitative and nucleotide-level map of 5′ of transcripts. Following the practice of the original study, the average output of the 3 bins in the center of sequence was used as the predicted expression for a sample. Each elements were also inserted into 600 random genome locations to compute the final averaged regulatory activity. this experiments was performed for 1,000 enhancers and 1,000 non-enhancer elements due to the time complexity of Enformer inference. The comparison with GET is performed on the same set of elements.

### Model Interpretation and Analysis

Calculation of jacobian matrix Multiple feature attribution methods were used in different analyses.

The gradient of the model's output with respect to the input features, represented by the vector ∇f(x), tells that how much the model output (Expression) will change when a small amount of the input along a dimension (e.g. a certain motif in a cisregulatory region) is changed.

The generalization to multiple outputs in the context of neural network feature attribution extends to the Jacobian matrix:

$${J}_{i , j} = \frac{\partial {f}_{i}}{\partial {x}_{j}} ,$$

where fᵢ is the i-th output, representing the transcription level on either the positive or negative strand, and xⱼ is the j-th input feature, comprising scanned and summarized binding scores for 282 TF motif clusters, and an additional dimension for accessibility scores.

For specific analysis, the input is a region-by-feature matrix of dimensions (200,283), including 282 features for TF motif clusters and 1 for accessibility scores. Two models are considered:
With-ATAC Model: Accessibility scores are set to the normalized TPM of Tn5 insertion count in the given accessible region.
Without-ATAC Model: Accessibility scores for all regions are set to 1, focusing solely on chromatin accessibility peaks.

The corresponding output is a region-by-strand matrix of dimensions (200,2), capturing the transcription levels on both positive and negative strands. This formulation enables the computation of the Jacobian matrix, vital for understanding the influence of individual features on the transcription levels.

Integrated Gradients (IG) provides importance scores by approximating the integral of gradients along a path from given baselines to inputs. The mathematical formulation is:

$$I G ( x ) = ( x - {x}_{baseline} ) \cdot {\int }_{0}^{1} ∇ f ( {x}_{baseline} + α ( x - {x}_{baseline} ) ) d α ,$$

where x_{baseline} is the baseline input.

DeepLIFT (Deep Learning Important FeaTures) attributes the difference in activation to each input feature, based on a reference input.

These methodologies offer unique perspectives on feature importance, with choices guided by computational efficiency, granularity, and the specific modeling context.

TABLE 3
Feature Attribution Approaches

| Methods | Conceptual Overview |
|---|---|
| Gradients/Jacobians | Provides a first-order approximation of feature |
| | influence on the output. |
| Integrated Gradients | Approximates the integral of gradients along a |
| | path, providing smoother and more detailed |
| | attribution, with baseline comparisons. |
| DeepLIFT | Focuses on differences in activation, running |
| | faster than Integrated Gradients, with support |
| | for specific non-linear activations. |

Identifying important regions and regulators Inference samples are first gathered across the genome by producing 200-region windows that centered around each genes promoter. Given a specific gene g on strand s∈{0,1}, the expression value can be inferred using the General Expression Transformer (GET) model f applied to an input matrix X∈ [symbol] ^{r×m} where r denotes the number of regions, and m includes motifs and optionally accessibility features:

$$E = f ( X )$$

$${E}_{g} = E [ r // 2 , s ]$$

where [⋅,⋅] is the indexing operator, s is the strand of the gene.

The jacobian matrix (tensor) J_{X} ∈R^{r×2×r×m} of f at the point (E,X) evaluates how each output dimension will change when each input dimension changes a small quantity. The output dimension and strand that correspond to the given gene are specifically picked, represented as ∇g∈ [symbol] ^{r×m}:

$$∇ g = {J}_{X} [ r // 2 , s ]$$

$${J}_{X} = \frac{\partial E}{\partial X}$$

The feature (motif) importance vector vg ∈ [symbol] ^{m} is obtained by multiplying the gradient element-wise with the original input and summarizing across regions:

$${v}_{g} = {\sum }_{i = 1}^{r} ( ∇ g ⊙ X ) [ i , : ]$$

Where ⊙ signifies the element-wise or Hadamard product. Since the gene-by-motif matrix is mostly used for feature-feature interaction analysis, the X with quantitative ATAC signal is used even when inferring Jx using a ‘Without-ATAC’ model. This helps to study the relationship between regulators and observed chromatin accessibility. The cell type c specific genome-wide gene-by-motif matrix V_{c} is acquired by concatenating the v_{g} across the genome. And the same process can be applied to different cell types.

Similarly, the region importance vector l_{g} ∈ [symbol] ^{r} is given by:

$${l}_{g} = {\sum }_{j = 1}^{m} ( ∇ g ⊙ X ) [ : , j ]$$

Regulator top targets Based on the gene-by-motif matrix V_{c}, a TF/motif (in this case, GATA) can be chosen and assessed for what genes will be mostly affected by this TF by identify the largest entries in the motif column. The top 1,000 genes were chosen and gene ontology enrichment analysis was performed using go:Profiler with the default “g_SCS” multiple hypothesis testing correction. To avoid general terms the result was filtered with term size (gene number in a term definition) larger than 500 and smaller than 1000. Terms with adjusted P-value smaller than 0.05 are retained as significant terms. TFs in the “Hemopoiesis” term with expression log₁₀TPM >1 were further selected for visualization against ‘GATA’ motif score.

Transcription Factor and Target Gene Correlation Analysis in Fetal Cell Types This analysis sought to elucidate the relationship between transcription factors (TFs) and their target gene expression across different cell types. Gene-by-motif files were aggregated and organized into a unified structure comprising genes, motifs, and corresponding cell features. The target genes for each TF were identified within predefined motif clusters, and the mean expressions of both the target genes and the corresponding TFs were computed. To avoid potential artifacts caused by experimental batch effect in expression measurement, the analysis was performed in both in adult+fetal cell types and also in only fetal cell types to get similar results.

The relationship was assessed using Spearman correlation analysis, with the results visualized through scatter plots. The x-axis represented the mean expression of the target genes, and the y-axis represented the mean expression of the TFs. Each plot was annotated with the Spearman correlation coefficient and the associated p-value, providing a statistical assessment of the correlation.

The analysis was performed iteratively for all TFs within the motif clusters specific to fetal cell types. The correlation coefficients and p-values were compiled, and the visualizations were saved as individual image files. This comprehensive view of the relationship between TFs and their target genes offers valuable insights into the regulatory dynamics within the context of fetal development.

Regulatory embedding The embedding of each gene was collected after each transformer block of GET. For a gene g, it's embedding is defined as the embedding vector of the promoter in the output of i-th block. The embedding contains not only promoter information but also information from surrounding regions owing to the attention mechanism. In general, the deeper the layer, the more its space is dominated by the expression output. UMAP was used to visualize the embedding. Louvain clustering was performed on the embedding space to colorize the UMAP visualization. Resolution is arbitrarily chosen to keep the cluster number around 10 and close to the UMAP density.

The embedding was computed in two different settings: cell type specific setting, where each dot is a gene embedding from a specific cell; cell type agnostic setting, where each dot is a gene embedding randomly sampled from all cell types. 50,000 embedding is sampled in the second case to make UMAP computation feasible.

Causal discovery of regulator interaction Pairwise Spearman correlation was performed using the gene-by-motif matrix also in both cell type specific and agnostic settings. Input*gradient score were used to constructed the matrix for computational efficiency. For the cell type agnostic settings, all genes with their promoter overlaps with an open chromatin peaks from all cell types are involved in the correlation calculation. Causal discovery was performed on the gene-by-motif matrix using LiNGAM. For the cell type agnostic settings, 50,000 genes were randomly sampled from all cell types, the resulting matrix were subjected to LiNGAM algorithm implemented in the Causal Discovery Toolbox python package.

To benchmark the predicted causal edges in the cell type agnostic setting, known physical interaction subnetwork from STRING V11 database was downloaded and interactions with a combined score larger than 400 were kept as the ground truth label. Since the pairs predicted by GET is on motif cluster level, the physical interactions between TFs onto the motif clusters were mapped based on the motif cluster annotation. The resulting motif-motif physical interaction network were then compared with the prediction to calculate the precision. All significant interactions determined by mass spectroscopy were also downloaded and compiled from the Human Transcription Factor protein interaction network and mapped to motif-motif interactions for comparison.

For the TF interaction database, the LiNGAM analysis was performed using cell-type-specific gene-by-motif table. Interactions with top 5% absolute effect size are retained in the final database. For each interaction, structural analysis between the two TFs with highest expression in the corresponding cell types was performed.

### Structural Analysis

Alphafold benchmark on intra-family binder prediction A TF was classified as an intra-family binder if any of its member TFs have a known physical interaction annotated in STRING V11 database. Based on the hypothesis that if a TF can bind as a heterodimer, due to sequence and structure similarity they should also have the potential of binding as a homodimer, although the dimerization affinity might be different. AlphaFold was used to predict the ‘hypothetical homodimer structure’ of all known TFs, and try to predict whether a TF could be an intra-family binder based on various AlphaFold-based metrics. Among several different Alphafold-based metrics, including mean_plddt (average predicted Local Distance Difference Test score across all residues), pAE (predicted Aligned Error across all inter-chain interactions), pDockQ (predicted DockQ metric using interface pLDDT), and pDockQ×pAE. pDockQ×pAE led to the best AUROC (0.69) and AUPR (0.41) when classifying intrafamily binder TFs.

Protein sequence segmentation pLDDT from Alphafold is a reliable protein domain caller due to its accurate structure prediction performance. Each TF protein sequence was segmented to low and high pLDDT regions. Empirically, it was found that 80% (recall) of known DNA-binding domains can be easily identified using high pLDDT regions plus high ratio of positive charged residues. More specifically, smoothed pLDDT is first computed using a 10 aa moving-average kernel and then normalize the score by dividing the max. After that, any region that has a smoothed pLDDT score less than 0.6 is defined as a low pLDDT region. If two low pLDDT regions are close (<30 aa) they are merged as one. Any region that is not a low pLDDT region are labeled as a high-pLDDT region.

Multimer structure prediction LocalColabFold and ColabFold is used to predict multimer structure with AlphaFold Multimer v2.3 model. For homodimer prediction, all 5 models were used with 3 recycles. For large scale interaction screening, model 3 and 3 recycles was used for each prediction. Predicted aligned error (PAE) and predicted LDDT were stored for downstream analysis. pDockQ were calculated following code from FoldDock.

For the large score interaction screening, exhaustive multimer prediction was performed between all possible low/highpLDDT segment pairs of the two protein in a pair. Then the new pLDDT of each segment in the multimer structure was compared with their original pLDDT in the monomer or homodimer structure.

Molecular dynamics simulation The initial configuration was prepared from the Alphafold predicted PDB file. The Amber99SB-dispersion (a99SBdisp) force field was employed for system parameterization. A cubic simulation box was defined with a box size of 1 nm. Subsequently, the system was solvated using the TIP4P water model through the solvate module. To neutralize the system and generate physiological ion concentrations, sodium (Na+) and chloride (Cl−) ions were added using the genion module. The energy minimization terminates upon reaching a maximum force below 1000.0 kJ/mol/nm. Each minimization iteration utilizes a step size of 0.01 and is configured to run for a maximum of 50,000 steps. The system was then equilibrated in two steps: first in the NVT (Constant Number, Volume, Temperature) ensemble and then in the NPT (Constant Number, Pressure, Temperature) ensemble for 100 ps of simulation time. A 100-ns production run was then performed and trajectories and energy profiles were stored for subsequent analysis. All configs of these are available at the Proscope repo.

Structure visualization ChimeraX was used to visualize the predicted structures. VMD were used to generate the movie of molecular dynamics simulation trajectory.

### Biological Experiments

Cell Culture HeLa cells were purchased from ATCC (CCL-2). HeLa cells were cultured in DMEM (Gibco, 11965) supplemented with 10% defined FBS (HyClone, SH30070), at 37° C./5% C02.

TFAP2A co-immunoprecipitation HeLa cell protein lysates were generated with 0.5% NP-40 lysis buffer (50 mM Tris-HCl, 150 mM NaCl, 0.5% NP-40) with phosphatase and protease inhibitor cocktail (Sigma-Aldrich, PPC1010). Samples were incubated with 5 μg agarose-conjugated TFAP2A primary antibody (Santa Cruz Biotechnology, sc-12726 AC) overnight at 4° C. Beads were washed, then boiled in Laemmli loading buffer (BioRad, 1610737). Proteins were separated on 10% Tris-Glycine gels (ThermoFisher, XP00100), transferred to PVDF (Immobilon-P, IPVH00010) and probed with primary antibodies against TFAP2A (ABclonal, A2294), ZFX (ThermoFisher, PA5-34376) and b-ACTIN (Santa Cruz Biotechnology, sc-47778) followed by chemiluminescence detection.

### Data Availability

Bulk RNA-sequencing of B-ALL Patients published in a previous study (Oshima, K. et al. Nat. Cancer 1, 1113-1127, (2020)) is available at SRA (PRJNA534488). Human transcription factor protein interaction networks are downloaded from supplementary data of G66s, et al (Göös, H. et al. Nat. Commun. 13, 766, 10.1038/s41467-022-28341-5 (2022)). Training data and trained model will be open sourced.

Precomputed regulatory inference result, preprocessed data and structure prediction can be viewed at GET website huggingface.co/spaces/get-foundation/GET. The full processed data and inference result will be provided in a public AWS S3 bucket.

### Code Availability

Code for pretraining, finetuning, data preprocessing, and results analyzing will be made available in github (github.com/RabadanLab, https://github.com/GET-Foundation). Pretrained model will be available on Huggingface (huggingface.co/get-foundation). Code for the website is available at huggingface.co/spaces/get-foundation/GET/tree/main.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A computer implemented method for identifying genetic regulatory modules, comprising:
obtaining sequence features of genomic regions and expression data for one or more target genes from a biological sample or database;
determining interaction between sequence features;
generating a score representing the effect on expression of a target gene for the sequence features or combinations thereof,
identifying regulatory modules based on the score; and
optionally, assigning a regulatory module as a transcriptional enhancer or transcriptional repressor based on the score.

2. The method of claim 1, wherein
the sequence features comprise chromatin accessibility data, transcription factor binding motif data, nucleotide sequences, or a combination thereof and/or
the expression data comprises bulk RNA-seq data, single cell RNA-seq data (scRNA-seq) or single nucleus RNA-seq data (snRNA-seq).

3. The method of claim 1, wherein the sequence features and/or expression data are derived from a single cell, a single cell type, bulk cell data, or a combination thereof.

4. The method of claim 1, wherein the genomic regions comprise regions: within 20 kilobases of the transcription start site of a target gene; greater than 1 Megabase from the transcription start site of a target gene; within 2 Megabases of the transcription start site of a target gene; or combination thereof.

5. The method of claim 1, wherein determining the interaction between sequence features comprises using a deep learning model.

6. The method of claim 5, wherein the deep learning model comprises a transformer model.

7. A computer implemented method for predicting target gene regulation and expression, comprising:
identifying regulatory modules for a target gene;
generating a score representing the effect on target gene expression for one or more identified regulatory modules with a machine learning model; and
optionally further comprising: generating a score for one or more modified regulatory modules, wherein the modified regulatory modules comprise a modification to one or more features of the regulatory modules; and/or designing, and optionally conducting, one or more gene editing experiments to generate a modified regulatory module.

8. The method of claim 7, wherein the modification comprises one or more nucleic acid substitutions, deletions, and/or insertions in a sequence of a regulatory module.

9. The method of claim 7, wherein training for the machine learning model is selected from the group consisting of unsupervised learning, self-supervised, semi-supervised learning, transfer learning and combinations thereof and wherein the training comprises:
obtaining sequence features of genomic regions and expression data for a plurality of target training genes from a single cell or cell line;
determining interaction between sequence features; and
identifying putative regulatory modules based on the effect on expression for the sequence features or combinations thereof,
wherein the target gene is from the same or different cell or cell-type as the single cell or cell line used in for training.

10. The method of claim 9, wherein
the sequence features comprise chromatin accessibility data, transcription factor binding motif data, nucleotide sequences, or a combination thereof and/or
the expression data comprises single cell RNA-seq data (scRNA-seq) or single nucleus RNA-seq data (snRNA-seq).

11. The method of claim 7, wherein the machine learning model comprises a language model.

12. The method of claim 7, wherein the genomic regions comprise: regions within 20 kilobases of the transcription start site of a target gene; within 2 Megabases of the transcription start site of a target gene; or a combination thereof.

13. The method of claim 7, wherein determining interaction between sequence features comprises using a deep learning model.

14. The method of claim 13, wherein the deep learning model comprises a transformer model.

15. A system comprising:
one or more processors; and
a non-transitory computer-readable medium storing instructions, that when executed by one or more processors performs operations to carry out the steps of the method of claim 1.

16. A system comprising:
one or more processors; and
a non-transitory computer-readable medium storing instructions, that when executed by one or more processors performs operations to carry out the steps of the method of claim 7.


SEQUENCE LISTING (2 sequences)

SEQ ID NO: 1 - DNA, 14 bp, synthetic construct
  source 1..14: mol_type = other DNA
taccgatcaactcg

SEQ ID NO: 2 - DNA, 13 bp, synthetic construct
  source 1..13: mol_type = other DNA
actccacgggatc